(19) United States
(12) Patent Application Publication
(10) Pub. No.: US 2026/0114750 A1
(43) Pub. Date: Apr. 30, 2026

(54) PLATFORM FOR FINGER MOTION ANALYTICS AND HEALTHCARE APPLICATIONS

(71) Applicant: THE PENN STATE RESEARCH FOUNDATION, University Park, PA (US)

(72) Inventors: Taiting Lu, University Park, PA (US); Hao Zhou, University Park, PA (US); Mahanth Gowda, University Park, PA (US); Yilin Liu, University Park, PA (US); Shijia Zhang, University Park, PA (US); Runze Liu, University Park, PA (US)

(73) Assignee: THE PENN STATE RESEARCH FOUNDATION, University Park, PA (US)

(21) Appl. No.: 19/474,098

(22) PCT Filed: May 7, 2024
(86) PCT No.: PCT/US2024/028113

§ 371 Date: Oct. 9, 2025

Related U.S. Application Data
(60) Provisional application No. 63/500,690, filed on May 8, 2023.

(51) Int. Cl. A61B 5/11; A61B 5/00; A61B 5/01

(52) U.S. Cl. CPC A61B 5/1114; A61B 5/0022; A61B 5/01; A61B 5/4845; A61B 5/6826; A61B 5/7267; A61B 5/742

(57) ABSTRACT

Methods and processes for activity tracking and health analytics applications can include a smart ring device comprising embedded sensors configured to collect user data. The collected data can be used to provide three-dimensional finger motion tracking analysis. In addition, or alternatively, the collected data can be used to provide blood alcohol concentration measurement and analysis.

### CROSS-REFERENCE TO RELATED APPLICATIONS

This patent application is related to and claims the benefit of priority of U.S. Provisional Application 63/500,690, filed on May 8, 2023, the entire contents of which is incorporated by reference.

### STATEMENT REGARDING FEDERALLY SPONSORED RESEARCH DEVELOPMENT

This invention was made with government support under Grant Nos. U.S. Pat. No. 2,046,972, CNS2008384 and CNS1909479 awarded by the National Science Foundation. The Government has certain rights in the invention.

### FIELD

The present disclosure generally relates to an apparatus comprising a smart ring device and, in particular, to methods and processes for activity tracking and health analytics applications via at least one smart ring device comprising embedded sensors. For example, some embodiments of the apparatus can be configured to provide three-dimensional finger motion tracking analysis. Some embodiments can further be configured to provide blood alcohol concentration measurement and analysis.

### BACKGROUND

Wearable devices such as a smart ring can be configured to communicate collected data from a user to an external device. Examples of these types of devices are disclosed in U.S. Pat. Nos. 11,291,378 and 11,922,809, as well as U.S. Patent Application Publication Nos. 2021/0089126, 2016/0113569, and 2023/0153416. However, we have determined that such devices and systems often include constraints, such as bulky form factors or a lack of wireless communication capabilities resulting in devices that may limit mobility and prevent use in everyday life.

### SUMMARY

We have determined that an apparatus can be designed to help overcome constraints included in previously existing smart rings. For instance, an apparatus comprising a wearable ring device with embedded sensors can be designed with the following requirements: (i) low form factor with small weight and size, (ii) longer battery life, (iii) wireless communication with an external device for streaming of sensor data for analysis, (iv) ability cover a wide array of sensing applications, (v) water resistance, and/or (vi) a curved display, such that a user can wear the ring all of the time. Embodiments can be configured to provide blood alcohol content measurements or other measurements related to the user being under the influence of a drug to help users avoid engaging in activities while under the influence of that drug (e.g. driving while under the influence of alcohol, etc.).

Embodiments of an apparatus and process can be configured to provide motion or activity tracking (e.g., three-dimensional finger motion tracking analysis) and/or healthcare analytics (e.g., blood alcohol concentration measurement and analysis). The apparatus can include a wearable ring device which can be adapted as a platform that can be worn on a user's finger. The device can be a type of communication device or computer device that includes hardware. The hardware can include a processor (e.g., microcontroller unit) connected to a non-transitory computer readable medium and one or more sensors and/or an array of sensors. The device can be communicatively connectable to one or more other devices via at least one network as well.

For example, some embodiments can be configured as a smart ring platform with IMU and PPG sensors for activity tracking and healthcare analytics applications. Embodiments can be configured to integrate the sensing units with a microcontroller unit and Bluetooth low energy (BLE) modules. The electronic components can be integrated on both sides of a flexible printed circuit board that is bent in the shape of a ring and enclosed in a flexible, waterproof housing for smooth skin contact. To validate the sensing capabilities in a prototype embodiment, we enabled an application in three-dimensional finger motion tracking. By extracting synthetic training data from public videos coupled with data augmentation to minimize the overhead of training data generation, the prototype embodiment was able to be configured to exploit correlations across fingers and time to track three-dimensional finger motion. We also validated the use of acquired PPG data from this prototype for heart rate monitoring. We further enabled an application in blood alcohol concentration (BAC) measurement and analysis. Ultimately, we believe embodiments of the device described herein can enable exciting applications in fitness tracking, metaverse, sports, healthcare, and other applications.

In an exemplary embodiment, a method of tracking three-dimensional finger motion comprises providing one or more wearable rings comprising a microcontroller unit, a non-transitory computer readable medium connected to the microcontroller unit, a Bluetooth transceiver unit connected to the microcontroller, and an inertial measurement unit (IMU) sensor; detecting motion of at least one of the one or more wearable rings via a motion detection module; collecting motion data via the IMU sensor; providing the motion data to a motion evaluation platform; processing the motion data using a motion determination processing mechanism implementing a trained machine learning model to generate a three-dimensional hand pose output.

In some embodiments, the trained machine learning model comprises a temporal encoder branch and a finger relation branch.

In some embodiments, the temporal encoder branch is trained by: feeding synthesized training data to the temporal encoder branch to generate a temporal output represented by the formula \hat{x}_{ti:ti-W}, wherein \hat{x} is the temporal output, tᵢ is any integer greater than or equal to one, and W is any empirically selected value.

In some embodiments, the synthesized training data is augmented prior to being fed to the temporal encoder branch.

In some embodiments, the finger relation encoder branch is trained by feeding synthesized training data to the finger relation encoder branch to generate a finger relation output represented by the formula \hat{f}_{ti:ti+W}, wherein f is the finger relation output, tᵢ is any integer greater than or equal to one, and W is any empirically selected value.

In some embodiments, the synthesized training data is augmented prior to being fed to the finger relation encoder branch.

In some embodiments, the temporal encoder branch and the finger relation encoder branch are fused to produce a final prediction of finger joint angles represented by the formula ŷ_{ti:ti+W}∈ [symbol] ^{18×W}, wherein ŷ is the final prediction of finger joint angles, tᵢ is any integer greater than or equal to one, and W is any empirically selected value.

In some embodiments, the trained machine learning model further comprises a loss function comprising a mean square error loss and a cosine similarity loss.

In some embodiments, the mean square error loss is represented by the formula

$${ℒ}_{mse} = {\sum }_{j = 1}^{j = 18} {( \hat{{ϕ}_{j}} - {ϕ}_{j} )}^{2} ,$$

wherein \hat{φ}ⱼ represents predicted finger joint angles and φⱼ represents joint angles extracted from synthesized training data.

In some embodiments, the cosine similarity loss is represented by the formula

$${ℒ}_{cosine similarity} = {\sum }_{j = 1}^{j = 18} \frac{\hat{{ϕ}_{j}} \cdot {ϕ}_{j}}{max ( ‖ \hat{{ϕ}_{j}} ‖ 2 \cdot ‖ {ϕ}_{j} ‖ 2 , ϵ )} ,$$

wherein \hat{φ}ⱼ represents predicted finger joint angles, φⱼ represents joint angles extracted from synthesized training data, and ε is 10⁻⁶.

In some embodiments, the method further comprises providing a first wearable ring, a second wearable ring, and a third wearable ring, each wearable ring comprising a microcontroller unit, a non-transitory computer readable medium connected to the microcontroller unit, a Bluetooth transceiver unit connected to the microcontroller, and an IMU sensor.

In some embodiments, the first wearable ring is position on a thumb of a user, the second wearable ring is positioned on an index finger of the user, and the third wearable ring is positioned on a ring finger of the user.

In some embodiments, the motion determination processing mechanism tracks three-dimensional finger motion with a joint accuracy of 6.57 mm.

In some embodiments, the motion determination processing mechanism tracks three-dimensional finger motion with a joint angle accuracy of 8.68 degrees.

In some embodiments, the motion detection module is hosted on an input/output device or a server.

In some embodiments, the one or more wearable rings further comprises an organic light-emitting diode display.

In some embodiments, the method further comprises displaying the motion data via the display to a user; displaying the three-dimensional hand pose output via the display to a user; or displaying messages configured to communicate information via the display to a user.

In an exemplary embodiment, a system for tracking three-dimensional finger motion comprises one or more wearable rings comprising a microcontroller unit, a non-transitory computer readable medium connected to the microcontroller unit, a Bluetooth transceiver unit connected to the microcontroller, and an IMU sensor, wherein the IMU sensor is configured to collect motion data; a motion detection module configured to detect motion of at least one of the one or more wearable rings; and a motion evaluation platform comprising a motion determination processing mechanism configured to process the motion data to generate a three-dimensional hand pose output, wherein the motion determination processing mechanism implements a trained machine learning model.

In some embodiments, the trained machine learning model comprises a temporal encoder branch and a finger relation branch.

In some embodiments, the temporal encoder branch is trainable via synthesized training data and configured to generate a temporal output represented by the formula \hat{x}_{ti:ti+W}, wherein \hat{x} is the temporal output, tᵢ is any integer greater than or equal to one, and W is any empirically selected value.

In some embodiments, the finger relation encoder branch is trainable via synthesized training data and configured to generate a finger relation output represented by the formula \hat{f}_{ti:ti+W}, wherein \hat{f} is the finger relation output, tᵢ is any integer greater than or equal to one, and W is any empirically selected value.

In some embodiments, the temporal encoder branch and the finger relation encoder branch are configured to be fused to produce a final prediction of finger joint angles represented by the formula ŷ_{ti:ti-W}∈ [symbol] ^{18×W}, wherein ŷ is the final prediction of finger joint angles, tᵢ is any integer greater than or equal to one, and W is any empirically selected value.

In some embodiments, the trained machine learning model further comprises a loss function comprising a mean square error loss and a cosine similarity loss.

In some embodiments, the mean square error loss is represented by the formula

$${ℒ}_{mse} = {\sum }_{j = 1}^{j = 18} {( \hat{{ϕ}_{j}} - {ϕ}_{j} )}^{2} ,$$

wherein \hat{φ}ⱼ represents predicted finger joint angles and φⱼ represents joint angles extracted from synthesized training data.

In some embodiments, the cosine similarity loss is represented by the formula

$${ℒ}_{cosine similarity} = {\sum }_{j = 1}^{j = 18} \frac{\hat{{ϕ}_{j}} \cdot {ϕ}_{j}}{max ( ‖ \hat{{ϕ}_{j}} ‖ 2 \cdot ‖ {ϕ}_{j} ‖ 2 , ϵ )} ,$$

wherein \hat{φ}ⱼ represents predicted finger joint angles, φⱼ represents joint angles extracted from synthesized training data, and ε is 10⁻⁶.

In some embodiments, the system further comprises a first wearable ring, a second wearable ring, and a third wearable ring, wherein the first wearable ring is positioned on a thumb of a user, the second wearable ring is positioned on an index finger of the user, and the third wearable ring is positioned on a ring finger of the user.

In some embodiments, the motion determination processing mechanism tracks three-dimensional finger motion with a joint accuracy of 6.57 mm.

In some embodiments, the motion determination processing mechanism tracks three-dimensional finger motion with a joint angle accuracy of 8.68 degrees.

In some embodiments, the motion detection module is hosted on an input/output device or a server.

In some embodiments, the one or more wearable rings further comprises an organic light-emitting diode display.

In some embodiments, the display is configured to display the motion data to a user; display the three-dimensional hand pose output to a user; or display messages configured to communicate information to a user.

In an exemplary embodiment, a method of monitoring blood alcohol concentration comprises providing one or more wearable rings comprising a microcontroller unit, a non-transitory computer readable medium connected to the microcontroller unit, a Bluetooth transceiver unit connected to the microcontroller, and a photoplethysmography (PPG) sensor; collecting light absorption data caused by changes of blood vessels via the PPG sensor;

providing the light absorption data to a BAC evaluation platform; and processing the light absorption data using a BAC determination processing mechanism implementing a trained machine learning model to generate a BAC estimation output.

In some embodiments, the BAC determination processing mechanism predicts blood alcohol centration with a mean absolute error of 0.0069.

In some embodiments, the BAC evaluation platform is hosted on an input/output device or a server.

In some embodiments, the one or more wearable rings further comprises an organic light-emitting diode display.

In some embodiments, the method further comprises displaying the BAC estimation output via the display to a user; or displaying messages configured to communicate information via the display to a user.

In an exemplary embodiment, a system for monitoring blood alcohol concentration comprises one or more wearable rings comprising a microcontroller unit, a non-transitory computer readable medium connected to the microcontroller unit, a Bluetooth transceiver unit connected to the microcontroller, and a photoplethysmography (PPG) sensor, wherein the PPG sensor is configured to collect light absorption data caused by changes of blood vessels; a BAC evaluation platform comprising a BAC determination processing mechanism configured to process the light absorption data to generate a BAC estimation output, wherein the BAC determination processing mechanism implements a trained machine learning model.

In some embodiments, the BAC determination processing mechanism predicts blood alcohol centration with a mean absolute error of 0.0069.

In some embodiments, the BAC evaluation platform is hosted on an input/output device or a server.

In some embodiments, the one or more wearable rings further comprises an organic light-emitting diode display.

In some embodiments, the display is configured to display the BAC estimation output to a user; or display messages configured to communicate information to a user.

In some embodiments, the system further comprises an inertial measurement unit (IMU) sensor and a thermal sensor.

In some embodiments, the IMU sensor is configured to collect motion data caused by changes in a user's activity or finger position.

In some embodiments, the thermal sensor is configured to collect skin temperature data.

### BRIEF DESCRIPTION OF THE DRAWINGS

The above and other objects, aspects, features, advantages, and possible applications of embodiments of the present innovation will be more apparent from the following more particular description thereof, presented in conjunction with the following drawings. Like reference numbers used in the drawings may identify like components.

FIG. 1 is a perspective view of an exemplary embodiment of a wearable ring.

FIG. 2 is an exploded view of the exemplary embodiment of the ring.

FIG. 3 is a perspective view of the exemplary embodiment of the ring. The ring is illustrated in a first position in which the ring can be worn by a user (e.g., around the user's finger).

FIG. 4 a schematic block diagram of a first exemplary circuit board arrangement for an exemplary embodiment of the ring.

FIG. 5 a schematic block diagram of a second exemplary circuit board arrangement for an exemplary embodiment of the ring.

FIG. 6 is a perspective view of the exemplary embodiment of the ring wherein the ring is connected to a docking station for charging.

FIG. 7A is a schematic block diagram illustrating an exemplary embodiment of a system for transmitting sensor data collected by a ring.

FIG. 7B is a schematic block diagram illustrating an exemplary embodiment of a system for transmitting sensor data collected by more than one ring.

FIG. 8 is a flow chart illustrating an exemplary embodiment of a system for transmitting sensor data collected by the ring.

FIG. 9 is a perspective view of the exemplary embodiment of the ring wherein the ring comprises a display.

FIG. 10 is a flow chart illustrating an exemplary embodiment of a system for tracking three-dimensional finger motion.

FIG. 11 is a graph illustrating real location data and synthetic location data extracted from the video training dataset under a common frame of reference.

FIG. 12 is a graph illustrating real orientation data and synthetic orientation data extracted from the video training dataset under a common frame of reference.

FIG. 13 is a graph illustrating motion enhancement of synthetic data via data augmentation.

FIG. 14 is a graph illustrating dynamic time warping of synthetic data via data augmentation.

FIG. 15 is a graph illustrating rotation augmentation of synthetic data via data augmentation.

FIG. 16 is a flow chart illustrating an exemplary embodiment of a machine learning model for tracking three-dimensional finger motion.

FIG. 17 is a flow chart illustrating an exemplary embodiment of a system for monitoring blood alcohol concentration.

FIG. 18 is a table illustrating the effectiveness of different components of a machine learning model for tracking three-dimensional finger motion.

FIG. 19 is a graph illustrating the results of a robustness study showing accuracy over fingers and wrists.

FIG. 20 is a graph illustrating the results of a robustness study showing accuracy over different users.

FIG. 21 is a graph illustrating the results of a robustness study showing accuracy degrading slowly with less sensors.

FIG. 22 is a graph illustrating the results of a robustness study showing error does not accumulate over long-duration and free-living experiments.

FIG. 23 is a table showing qualitive tracking results demonstrating close tracking of hand poses without placing sensors on all fingers.

FIG. 24 is a graph illustrating exemplary PPG sensor data processed by a bandpass filter.

FIG. 25 is a graph illustrating estimated heart rates during various activities.

FIG. 26 is a graph illustrating heart rate monitoring accuracy across individuals.

FIG. 27 is a flow chart illustrating an exemplary embodiment of a convolutional neural network of a machine learning model for blood alcohol concentration monitoring.

FIG. 28 is an exploded view of the exemplary embodiment of the ring.

FIG. 29 is a cross-section view of an exemplary embodiment of the ring being worn by a user.

FIG. 30 a schematic block diagram of a third exemplary circuit board arrangement for an exemplary embodiment of the ring.

### DETAILED DESCRIPTION

The following description is of exemplary embodiments and methods of use that are presently contemplated for carrying out the present invention. This description is not to be taken in a limiting sense, but is made merely for the purpose of describing the general principles and features of various aspects of the present invention. The scope of the present invention is not limited by this description.

A smart ring is an electronic, wearable device that can be worn around a user's fingers and has sensing, processing, and networking capabilities that allow the ring to interact with the user and/or other devices. For example, the ring described herein comprises at least one embedded sensor to collect sensor data. This data can include physiological and motion data and can be transmitted to an external device such that the data may then be stored, tracked, analyzed, etc. The ring described herein may therefore be employed in a variety of industries and applications wherein collection of data may be necessary or advantageous. For example, the smart ring described herein may be used in health monitoring applications (e.g., heart rate, blood pressure, blood alcohol concentration, etc.), activity monitoring (e.g., daily life activities, running, eating, drinking, sleep cycles, etc.), finger motion tracking (e.g., sign language translations), among other applications.

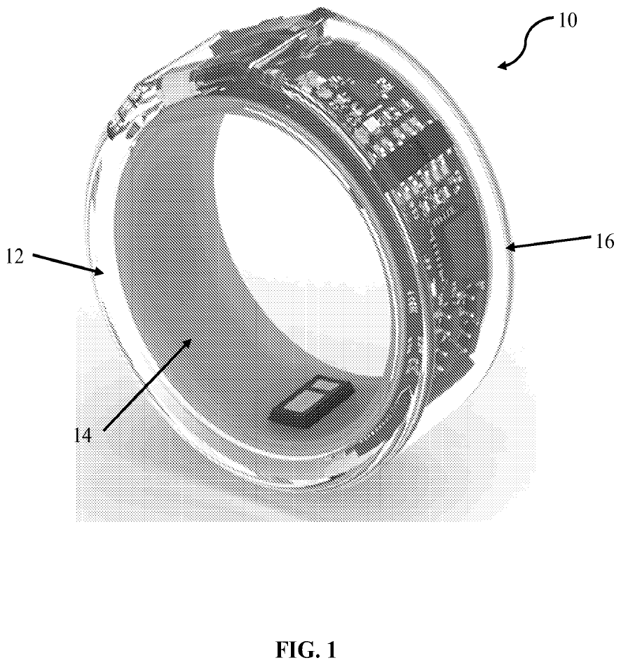

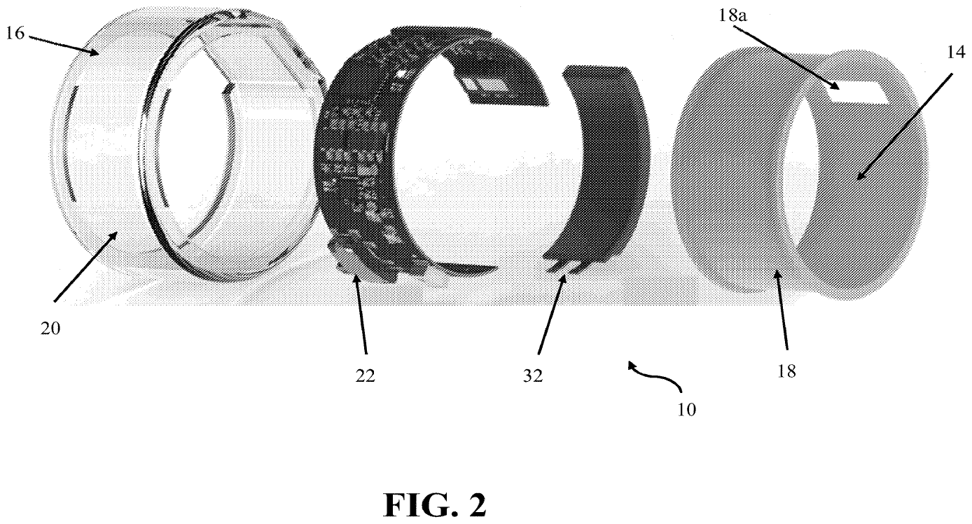

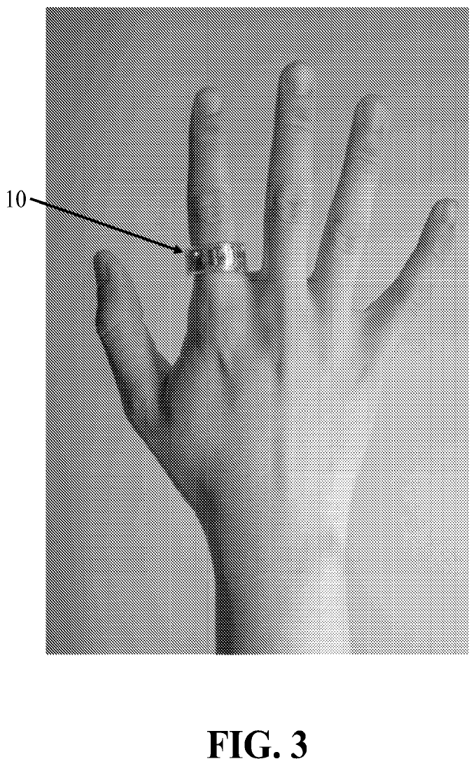

Referring to FIGS. 1-3 and 28, embodiments of an apparatus can be configured as at least one wearable ring 10, or as a system that utilizes at least one wearable ring 10, that is structured as an electronic device. The ring 10 is adjustable between a first position in which the device is worn by a user (e.g., around a user's finger; see FIG. 3) and a second position in which the device is separated from a user (e.g. taken off a user's finger).

Embodiments of the ring 10 disclosed herein advantageously have a small form-factor (e.g., size and configuration). For example, the ring 10 may have a weight of 2.5-2.9 g (without display 38) or 5.6-6.6 g (with display 38). It is contemplated that the weight of the ring 10 may depend on numerous factors, such as a user's finger size/diameter, specific components included within the ring 10, etc. The ring 10 may further have an average thickness of 3.8-4.0 mm, and may further have a width of 10 mm (without display 38) or 18.2 mm (with display 38).

The ring 10 has a housing 12 forming an annularly shaped profile having an inside surface 14 and an outside surface 16. The inside surface 14 defines an inner diameter of the ring 10 and the outside surface 16 defines an outer diameter of the ring 10. The inside surface 14 is configured to contact the user when the ring 10 is in the first position, and the outside surface 16 is configured to be exposed and visible to the user when the ring 10 is in the first position.

The housing 12 may comprise an interior cover 18 and an exterior cover 20, wherein the interior cover 18 is couplable to the exterior cover 20 such that the interior cover 18 and the exterior cover 20 define an encapsulated region. The interior cover 18 comprises the inside surface 14 of the ring 10, and the exterior cover 20 comprises the outside surface 16 of the ring 10. The term “encapsulated region” means an area that is enclosed (e.g., surrounded on all sides) such that the area is not exposed to surroundings. It is contemplated that the interior cover 18 may be coupled to the exterior cover 20 via a snap connection, press fit connection, welding, or any other suitable coupling. It is further contemplated that the interior cover 18 may be removably coupled to the exterior cover 20. In alternative embodiments, the interior cover 18 and the exterior cover 20 may be formed as a single molded piece.

The interior cover 18 and/or the exterior cover 20 may be formed from a polymeric material. It is contemplated that the interior cover 18 and/or the exterior cover 20 may be formed from an optically transparent or substantially transparent material (e.g., polymeric material, glass, crystal, etc.). Alternatively, the interior cover 18 and/or the exterior cover 20 may be formed from an opaque or colored material. It is further contemplated that the polymeric material may be flexible. In a preferred embodiment, at least the interior cover 18 is made from a flexible material, as a flexible material may be advantageous for sensing applications in which proper contact pressure with a user is needed. The polymeric material may further be waterproof and/or water resistant. In a preferred embodiment, both the interior cover 18 and the exterior cover 20 are made from a waterproof and/or water resistant material, therefore allowing the ring 10 to be safely worn during environmental conditions such as rain and snow, and during activities such as sweat-inducing activities, water sports, swimming, bathing, etc.

In a preferred embodiment, the polymeric material comprises thermoplastic polyurethane (TPU). It is contemplated that TPU can provide smooth contact with the user's skin, thus allowing long-term and comfortable wearing, while also providing lightweight and waterproof properties. In an alternative embodiment, the polymeric material comprises polylactic acid (PLA). It is contemplated that PLA provides a sturdy material that may protect components included within the encapsulated region of the ring 10. Other embodiments may utilize other suitable materials (e.g. other type of polymeric material, etc.).

It is contemplated that the interior cover 18 and the exterior cover 20 may be formed from the same material. Alternatively, the interior cover 18 and the exterior cover 20 may be formed from different materials.

The ring 10 includes a flexible circuit board 22 positioned within the housing 12, such as within the encapsulated region defined by the interior cover 18 and exterior cover 20. It is contemplated that a flexible circuit board allows the circuit board 22 to bend in the form factor of the ring 10. The circuit board 22 comprises a plurality of electrical components, such as a microcontroller unit 24 that is connected to non-transitory memory 26 or other type of non-transitory computer readable medium, a Bluetooth transceiver unit 28, and a sensor array 30 comprising at least one sensor. It is contemplated that the circuit board 22 may have a first side and a second side opposite of the first side. It is further contemplated that electrical components may be printed on both the first side and the second side of the circuit board 22. Printing electrical components on both sides of the circuit board 22 contributes to the small form factor of the ring 10.

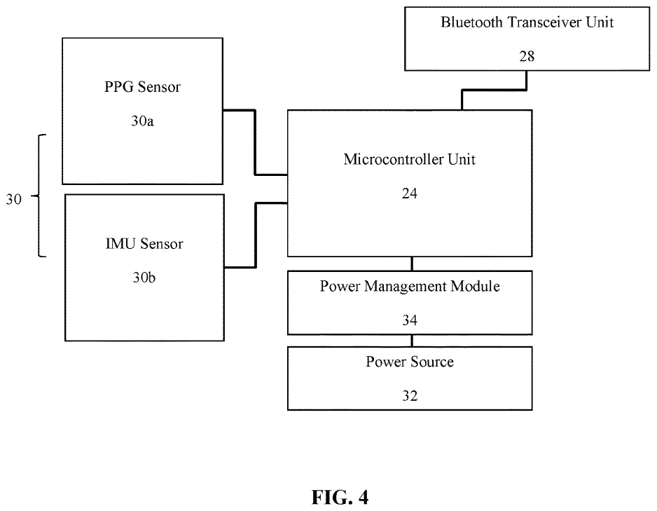

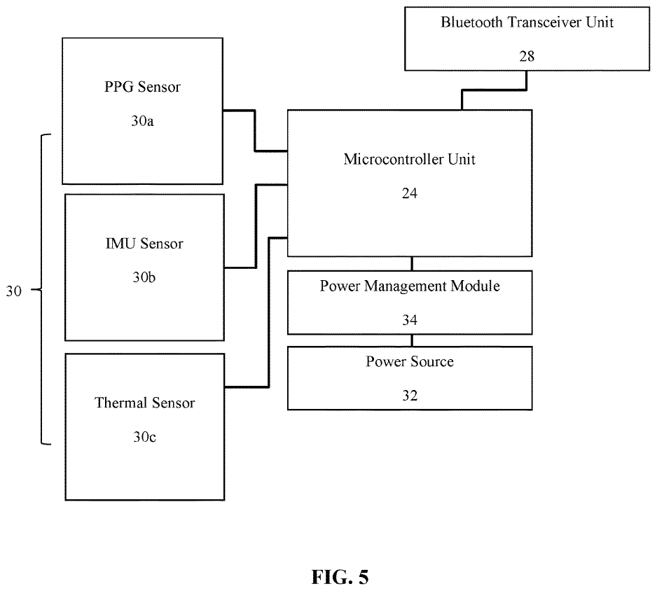

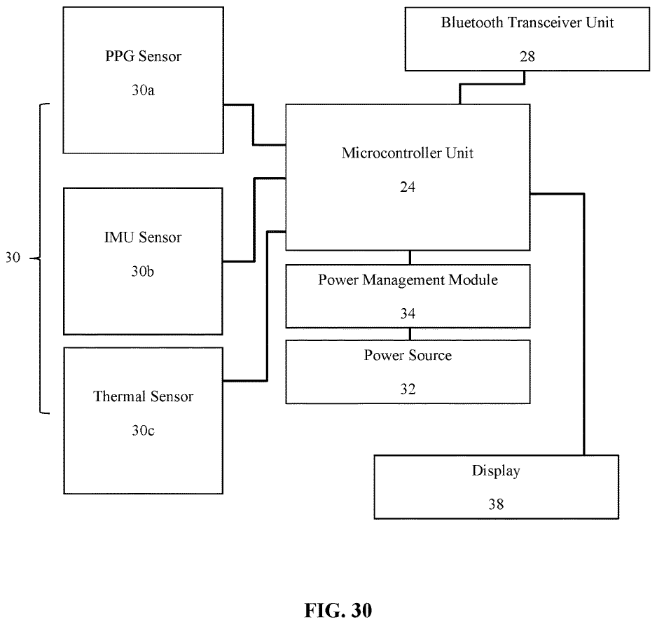

As seen in FIGS. 4-5 and 30, the microcontroller unit 24 interfaces with the rest of the components of the circuit board 22. A power source 32 can also be positioned in contact with the circuit board 22 for powering the circuit board 22, the microcontroller 24, and electric components thereof.

The power source 32 provides a source of power to the circuit board 22 and its components. The power source 32 can be a rechargeable battery or other type of battery, for example, that can provide a source of voltage to power the ring 10 and the components of the ring 10. In a preferred embodiment, the power source 32 is a lithium battery. The power source 32 can also include a power management module 34 that can include a low dropout regulator (LDO) as well as contact charging and battery charging modules configured to facilitate different charging mechanisms for charging the battery (e.g. charging via UBS connection, wireless charging, magnetic resonant coupling for charging of the battery, etc.).

In an exemplary embodiment, the power source 32 may be positioned within the housing 12 and/or on a surface of the housing 12. In a preferred embodiment, the power source 32 can be positioned at or protruding through an aperture 18a of the interior cover 18 of the housing 12. For example, as will be discussed in more detail below, the power source 32 can include terminals positioned at or protruding through an aperture 18a of the interior cover 18 to facilitate connection to a recharging device of a docking station 36 for receipt of electrical current for recharging of the power source.

In an exemplary embodiment, the power source 32 may be an arc-shaped (e.g., curved) battery. For example, the power source 32 may be curved to be compatible with the annularly shaped profile of the housing 12. In this embodiment, the flexible circuit board 22 may similarly be curved to be compatible with the annularly shaped profile of the housing 12, such that the power source 32 and the circuit board 22 together may match the entire annular shape of the housing 12. It is contemplated that by integrating the shape of the power source 32 into the overall shape of the ring 10, the overall form factor of the ring 10 may be reduced.

The ring 10 can have a power switch or power button (not shown) defined on the housing 12 or have another mechanism for being activated (e.g. detection of being in contact with skin to automatically turn on the ring 12, etc.).

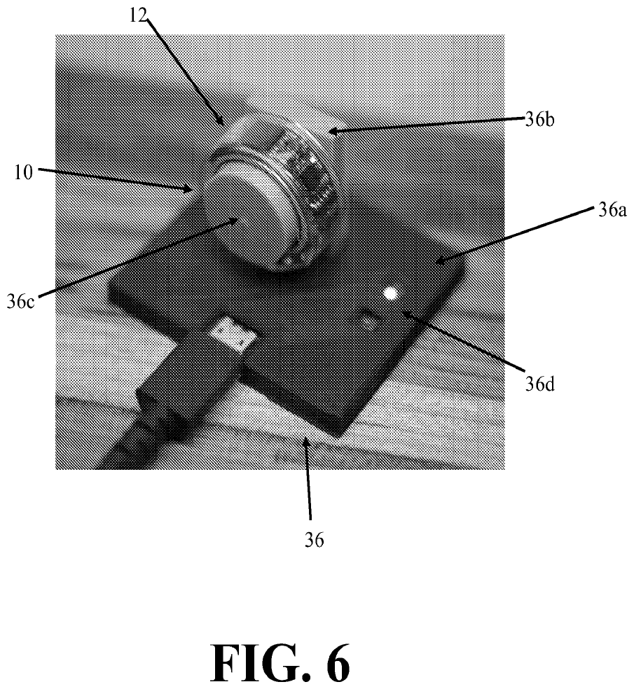

As seen in FIG. 6, the housing 12 can be configured to be communicatively connectable to a docking station 36. In some embodiments, the docking station 36 can be configured as a recharging station for the ring 10 that can recharge the power source 32 of the ring 10. For example, as discussed above, the power source 32 can include a terminals positioned at or protruding through an aperture 18a of the interior cover 18 to facilitate connection to a recharging device of a docking station 36 for receipt of electrical current for recharging of the power source.

In some embodiments, the docking station 36 can include a base 36a, a pillar 36b extending vertically from the base 36a, and a mounting projection 36c extending horizontally from the pillar 36b and towards a central portion of the base 36a. It is contemplated that an inner diameter of the ring 10 may be larger than the diameter of the mounting projection 36c such that the ring 10 may be positioned on and encircle the mounting projection 36c.

In some embodiments, an upper portion of the mounting projection 36c may have receiving terminals projecting from an upper surface thereof. In a preferred embodiment, the receiving terminals are easily visible to a user of the docking station 36. It is contemplated that such receiving terminals may complement the terminals at or protruding through an aperture 18a of the interior cover 18 of the ring 10. The receiving terminals may therefore facilitate a connection between the between the ring 10 and the docking device 10 and may help further ensure the ring 10 is properly oriented on the docking station 36 to allow for charging.

The docking station 36 can also include a light emitting device 36d that can be flashed or illuminated in a first color to indicate the power source 32 of the ring 10 is being charged and flashed or illuminated in a second color that differs from the first color to indicate when charging of the power source 32 is completed.

As described above, the circuit board 22 can have a sensor array 30 comprising at least one sensor. The at least one sensor is configured to collect sensor data. Each of the sensors of the sensor array 30 can be configured to detect or measure at least one physical property of a user and collect corresponding data. The sensors of the sensor array 30 can be communicatively connected to the microcontroller unit 24 and/or the non-transitory computer readable medium such that the sensors interface with and provide data to the microcontroller unit 24 and/or the non-transitory computer readable medium (e.g., via a serial peripheral interface (SPI), an inter-integrated circuit (I²C), a universal asynchronous receiver-transmitter (UART), etc.)

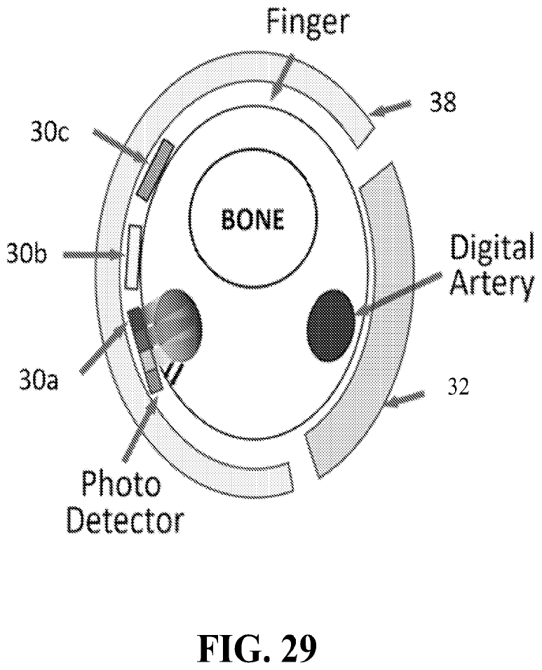

The sensor array 30 may comprise at least one photoplethysmography (PPG) sensor 30a. It is contemplated that a PPG sensor is an optical measurement method that uses light reflection data to measure various physiological properties. For example, a PPG sensor typically includes at least one light emitting diode that emits light and a photodetector that detects the intensity of reflected light (e.g., after the emitted light is reflected back to the photodetector) (see FIG. 29). Accordingly, the PPG sensor 30a may be configured to detect and collect data such as heart rate data, blood pressure data, blood oxygen saturation (SpO₂) data, blood alcohol concentration (BAC) data, and/or other metrics of interest, and any combination thereof. The PPG sensor 30a may interface with and provide data to the microcontroller unit 24.

It is further contemplated that the sensor array 30 can comprise at least one inertial measurement unit (IMU) sensor 30b. The IMU sensor 30b may be configured to detect and collect data from a user, such as motion data, position data, and/or other metrics of interest, and any combination thereof. The IMU sensor 30b may interface with and provide data to the microcontroller unit 24.

In one embodiment, the sensor array 30 only comprises a PPG sensor 30a and an IMU sensor 30b, However, the sensor array 30 may comprise other sensors 30c (e.g., a thermal sensor, an electrocardiogram (ECG) sensor, and/or an ultrasonic sensor). For example, a thermal sensor may be configured to measure the internal body temperature of a user wearing the ring 10. In another embodiment, the sensor array 30 only comprises a PPG sensor 30a, an IMU sensor 30b, and a thermal sensor 30c. In yet other embodiments, the sensor array 30 includes multiple sensors in addition to the PPG sensor 30a, IMU sensor 30b, and thermal sensor 30c.

Any number of the sensors of the sensor array 30 may be provided as a chip coupled to the microcontroller 24, such that the microcontroller 24 and circuit board 22 incorporates system-on-chip (SoC) architecture. It is contemplated that such SoC architecture contributes to a microcontroller 24 and circuit board 22 with a small form factor.

Embodiments of the apparatus can be configured as at least one ring 10, such as a plurality of rings 10. For example, in embodiments including a plurality of rings 10, each ring 10 may be worn on a different finger of the user, and the sensors of the sensor array 30 of each ring 10 may independently collect sensor data. In some embodiments, the apparatus may include at least three rings 10, and it is preferred that in such embodiments a user wears a first ring 10 on the user's thumb, a second ring 10 on the user's index finger, and a third ring 10 on the user's forefinger, middle finger, or pinky so that all the rings are worn on fingers of the same hand. In yet other embodiments, the rings can be positioned on other fingers on the same hand.

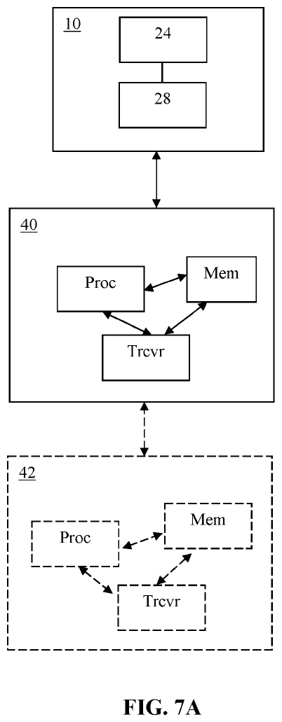

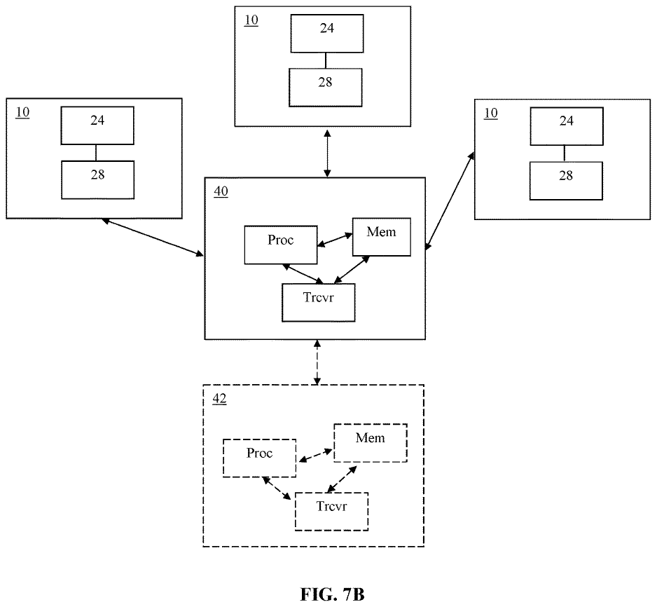

The data collected by the sensor array 30 of each ring 10 can be assembled (e.g., packaged into a packet of data) by the microcontroller unit 24, and the microcontroller unit 24 may then transmit the data wirelessly to an input/output device 40 (e.g. a smart phone, tablet, laptop computer, personal computer, etc.) using the Bluetooth transceiver unit 28 (see FIGS. 7A and B). While FIGS. 7A and 7B are schematic block diagrams including one ring 10 and three rings 10, respectively, as explained above the disclosure encompass all embodiments with at least one ring 10 (e.g. use of two rings on the same hand, use of four rings worn on four different fingers of the same hand, use of size rings in which three rings are positioned on different fingers of each hand, use of a single ring or multiple rings on different hands, etc.). The Bluetooth transceiver unit 28 can be configured as a wireless interface for the transmission of sensor data via each ring's wireless connection with an input/output device 40. For example, the Bluetooth transceiver unit 28 can be a Bluetooth Low Energy (BLE) system-on-chip coupled to the microcontroller 24 to facilitate Bluetooth wireless communications with the input/output device 40. The Bluetooth transceiver unit 28 can also have an antenna that is connected to the microcontroller 24 and helps facilitate the transmission of sensor data.

In some embodiments, the data collected by the sensor array 30 of each ring 10 may be continuously streamed to the input/output device 40. In other embodiments, the data collected by the sensor array 30 of each ring 10 may be periodically streamed to the input/output device 40 (e.g., non-continuously at pre-determined intervals). The data collected by the sensor array 30 of each ring 10 can also be stored within the non-transitory computer readable medium of the ring 10 for an extended period of time before being transmitted to the input/output device 40.

It should be appreciated that the input/output device 40 is a computer device that has at least one processor (Proc) connected to at least one non-transitory computer readable medium (e.g. memory, a hard drive, etc.) (Mem) and at least one transceiver unit (e.g. a network transceiver, etc.) (Trcvr).

As can be appreciated from the above, each ring 10 can be configured to facilitate transmission of data to at least one input/output device 40. It is contemplated that each ring 10 may transmit data to at least one input/output device 40 in response to activation of the ring 10. In response to being activated, the Bluetooth transceiver unit 28 of each ring 10 can output a transmission to at least one input/output device 40 to advertise its availability for connection for forming a wireless communication connection with a desired input/output device 40. The input/output device 40 may have an application stored in their memory that can be executed by a processor of the device to facilitate automatic connection with each ring 10 in response to the connectivity advertisement transmission output from each ring 10. In an event where a ring 10 had not previously formed a secure authorized connection, it may respond to the availability of the input/output device 40 by requesting and/or receiving the MAC address of the device to which the ring 10 is to connect for establishing the communicative connection with that device.

If a ring 10 is connected to the input/output device 40, the sensor data collected by the sensor array 30 can be sent to the device. The data being transmitted to the input/output device 40 may be stored data or a continuous stream of current data. The data may be collected continuously or at pre-selected monitoring intervals (e.g. every five seconds, every minute, etc.). Each sensor of the sensor array 30 may collect data continuously or discretely within pre-selected sensor measurement intervals spaced apart from each other by a pre-selected monitoring interval time period.

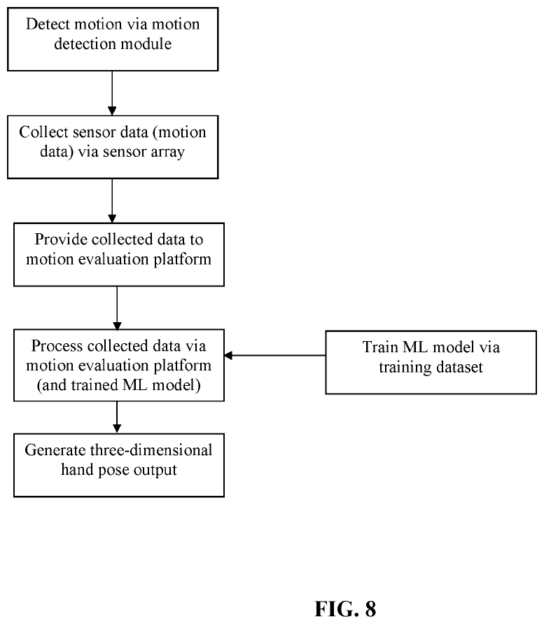

FIG. 8 illustrates an exemplary method by which the sensor data collected by the sensors of the sensor array 30 of each ring 10 can be processed for storage and/or transmission. For instance, the sensor data can be obtained from sensors of the sensory array 30 via at least one sensor interface (e.g., via a serial peripheral interface (SPI), an inter-integrated circuit (I²C), a universal asynchronous receiver-transmitter (UART), etc.) that connects the sensors to the microcontroller unit 24. The sensor data can be processed via the sensor interface. For example, the sensor data may be packaged as a data packet. The processed data can then be transmitted for secure storage and/or stored on the computer readable medium of the ring 10. When the stored sensor data is transmitted to the input/output device 40, transmission of related data can be included with the transmitted sensor data to identify the transmission process used to transmit the sensor data, time of transmission, time at which sensor data was stored and/or collected, sensor device identifier for each type of sensor data, as well as other data relevant to the collection and storage of the sensor data for subsequent evaluation by the input/output device 40 and/or other computer device.

It is contemplated that the data may alternatively or subsequently be sent to a server 42 that can be connected to the input/output device 40 (e.g. via a network connection and application programming interface (API) the server may have with the input/output device 40 running the application, etc.). The server 42 may be configured to store the sensor data of each ring 10. The forwarding of such data can be provided via a communication connection the ring 10 directly has with an input/output device 40 such that data is sent to the server 42 via the input/output device 40.

It should be appreciated that the server 42 can be configured as a computer device that has at least one processor (Proc) connected to at least one non-transitory computer readable medium (e.g. memory, a hard drive, etc.) (Mem) and at least one transceiver unit (e.g. a network transceiver, etc.) (Trcvr). The server 42 can be configured as a database server, for example. The server 42 can also be configured as a host server that hosts services provided to the user of the ring 10 via communications exchanged between the server 42 and the at least one ring 10, and/or at least one input/output device 40.

It is contemplated that once the data is transmitted to the input/output device 40 and/or the server 42, the data may be analyzed and evaluated. For example, the data can be processed using artificial intelligence or machine learning algorithms stored on the input/output device 40 and/or the server 42. In particular, the input/output device 40 and/or server 42 may run a program that uses the collected sensor data along with a module trained via a machine learning process that received the collected sensor data and processes that data to determine an output (e.g., user activities).

In some embodiments, the input/output device 40 and/or the server 42 may comprise a graphical user interface (GUI) for providing information to the user regarding collected sensor data. For example, the at least one ring 10 may transmit data collected from the sensor array 30 to the input/output device 40 and/or the server 42, and the collected data may be displayed in the form of a graph, chart, text, or other display via the GUI. The GUI may further display outputs or other collected data to the user (e.g., types of activities, duration of activities, BAC content, etc.).

Preferred embodiments of the ring 10 disclosed herein are configured to include a low power microcontroller 24 and low power electrical components, as these allow for an elongated period of time in which the power source 32 may provide a source of power to the ring 10 before requiring recharging. In some embodiments, the power source 32 may support up to a week of battery life. This is advantageous and convenient as it allows a user to wear the ring 10 at all times, including while sleeping.

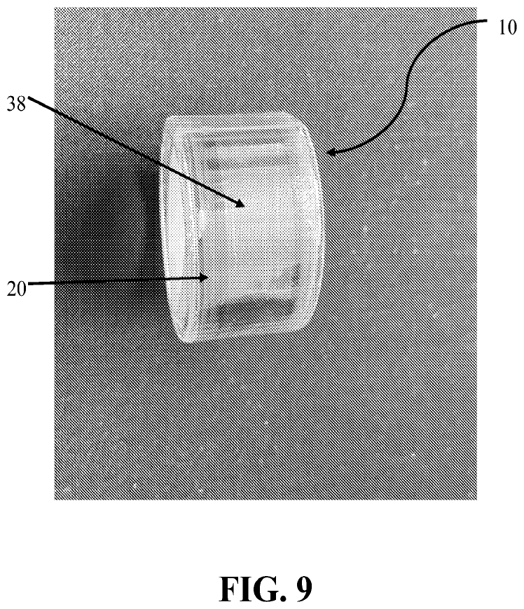

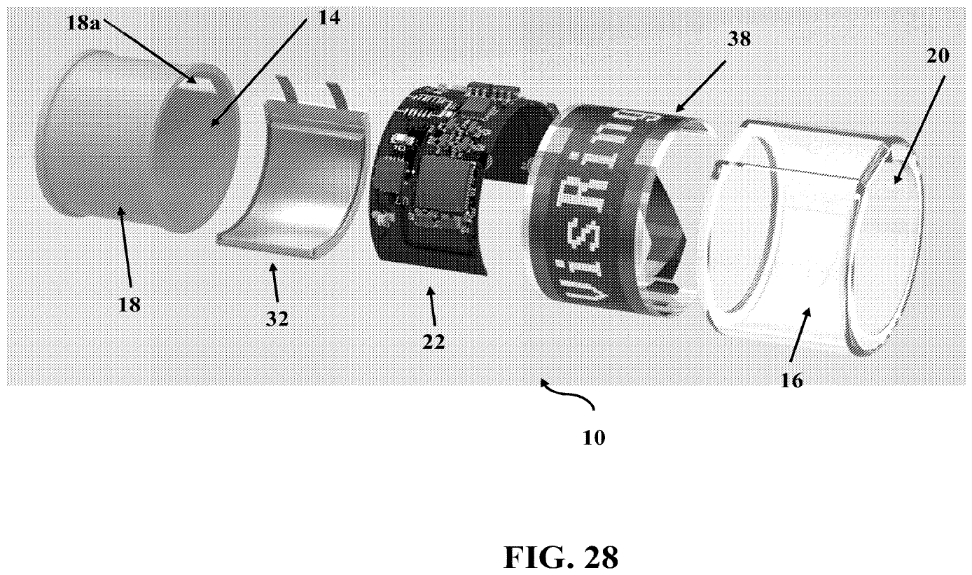

In some embodiments, as seen in FIG. 28, the ring 10 may further comprise a display 38 (see FIG. 9). The display 38 can be communicatively connected to the microcontroller unit 24 and/or the non-transitory computer readable medium such that the display 38 interfaces with the microcontroller unit 24 and/or the non-transitory computer readable medium (e.g., via a serial peripheral interface (SPI)). The display 38 may be a liquid crystal display (LCD), an organic light-emitting diode (OLED) display, or any other type of display. It is contemplated that the display 38 is a flexible display, such that may bend in the shape of the ring 10. In embodiments wherein the display 38 is positioned within the housing 12, it is contemplated for at least the exterior cover 20 to be formed from an optically clear or substantially clear material such that a user can see the display 38 through the housing 12 (see FIGS. 9 and 28). The display 38 can be configured to be de-activatable to reduce power consumption.

The display 38 can be configured to communicate data to the user. For example, the ring 10 can communicate collected sensor data for presentation on the display 38 of the ring 10. The display 38 may also be configured to communicate messages to the user.

In some embodiments, the display 38, the input/output device 40, and a sensor from the sensor array 30 may together be configured to solicit input from the user. For example, a user can be asked a question that prompts an answer from the user, such as “Answer Phone Call?”, via the display 38. In this example, there may be predetermined movements that correspond to certain responses—movement of the hand/finger comprising the ring 10 to the left may correspond to a “No” answer (e.g., an incoming phone call on the input/output device 40 will not be answered), or movement of the hand/finger comprising the ring 10 to the right may correspond to the a “Yes” answer (e.g., an incoming phone call on the input/output device 40 will be answered). It is contemplated that the IMU sensor 30b may be configured to detect motion of the finger/hand and the user's input to the solicitation. It is understood that this is only one example of how the sensor array 30, display 38, and/or input/output device 40 may work in conjunction to solicit input from a user, and this disclosure is by no means limited to this example.

### Three-Dimensional Finger Motion Tracking

As explained above, sensor data may be transmitted from at least one ring 10 to the input/output device 40 and/or server 42 for analysis, evaluation, etc. For example, it is contemplated that a user may wear a single ring 10 on one of the user's fingers. In such embodiments, the IMU sensor 30b may track the position and motion of a user's ring, and the microcontroller 24 may transmit this data to the input/output device 40 and/or server 42. It is therefore contemplated that this data may be used to track the motion of the user's finger on which the ring 10 is being worn.

In exemplary embodiments, sensor data may be used to track the finger motion of all fingers of a user (e.g., three-dimensional finger motion tracking). However, there are challenges associated with tracking finger motion of more than one finger. For example, the search space for three-dimensional finger pose is large (24 degrees of freedom (DoF)) and tracking these without rings/sensors on all fingers is an under-constrained problem without closed-form equations. To address this issue, we developed a learning based approach that exploits the inter-finger correlations to fill the gaps in sensing and automatically learns the mapping between sparse sensor data and three-dimensional finger motion.

In one embodiment, a user may wear a first ring and a second ring. In a preferred embodiment, a user may wear a first ring, a second ring, and a third ring (see, e.g., FIG. 7B). In a most preferred embodiment, a first ring may be worn on the user's thumb, a second ring may be worn on the user's index finger, and a third ring may be worn on the user's annular (e.g., ring) finger. In particular, it is contemplated that by wearing three rings, three-dimensional finger motion of all finger joints may successfully be tracked without wearing rings on each finger by exploiting correlations across fingers and time.

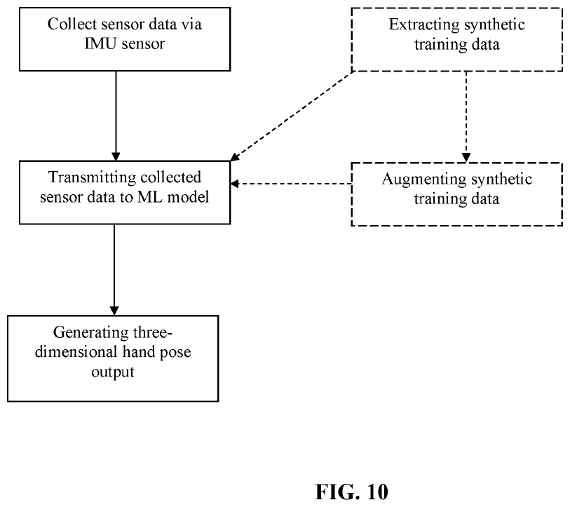

As seen in FIG. 10, an exemplary method of tracking three-dimensional finger motion therefore includes capturing sensor data using the IMU sensor 30b of each ring 10. It is contemplated that the sensor data may include at least location/position data, orientation data, and motion data from each ring.

It is contemplated that the method may first include detecting motion of at least one ring 10. For example, a motion detection module may be configured to detect the motion of at least one ring 10. The motion detection module may then provide motion data (e.g., sensor data captured using the IMU sensor 30b of each ring) based on the detected motion. The motion detection module may be hosted on an input/output device 40 and/or a server 42.

The method further includes transmitting the motion data to a motion evaluation platform. The motion evaluation platform may be configured to process the collected motion data to generate a three-dimensional hand pose output based on the sensor data from the ring(s) communicated to that platform. For example, the motion evaluation platform may include a pre-defined motion determination processing mechanism to process the collected motion. The motion evaluation platform may be hosted on an input/output device 40 and/or a server 42.

In some embodiments, the pre-defined motion determination processing mechanism may implement a trained machine learning (ML) model. The ML model may be hosted on an input/output device 40 (e.g. a smart phone, a laptop computer, a tablet, etc.) and/or a server 42 that may be communicatively connected to the input/output device 40 (e.g. via an application programming interface, API, or other communicative connection that may be provided via at least one network such as the internet and/or a local area network (LAN), etc.). It is contemplated that the ML model uses data together with machine learning algorithms that undergo a training process to prepare a trained ML model. In this case, the ML model may use a predetermined training dataset and machine learning algorithms to prepare a trained ML model that may utilized by the motion evaluation platform in processing collected motion data to generate a three-dimensional hand pose output.

Machine learning algorithms are typically trained with a training dataset. Accordingly, to train the ML model of the motion evaluation platform, locations of finger joints and wrists (location data) and orientation of the finger joints and wrists (orientation data) may be extracted from a video training dataset. In a preferred embodiment, the video training dataset may include intricate and/or diverse finger motions by multiple different users, as such finger motions (e.g., high complexity and speed) may subsume finger motions seen in applications that are more simplistic. However, there are no large-scale high-quality training datasets for three-dimensional finger motion tracking, and creating such datasets is expensive and time-consuming. Accordingly, we addressed this issue by harvesting a synthetic training dataset from publicly available videos and extracting necessary data from the dataset.

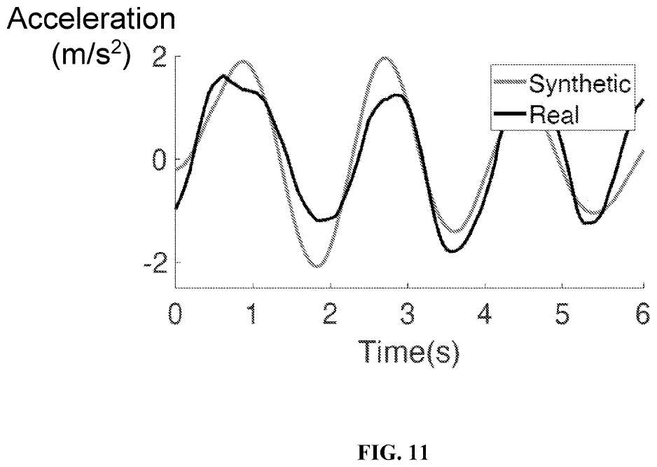

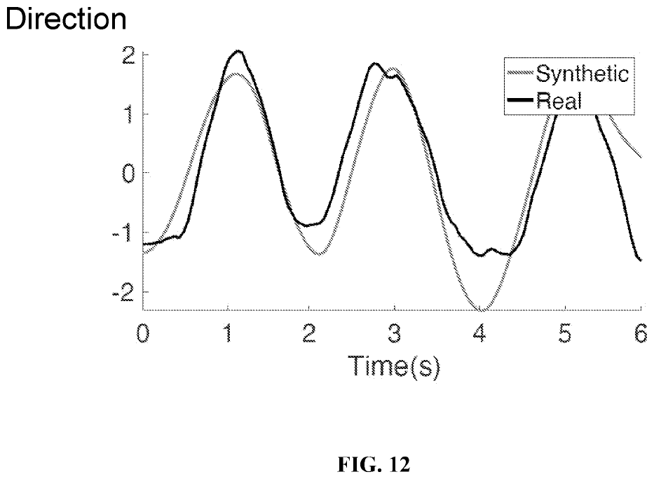

Once the location and orientation data is extracted, they may be used to train the machine learning algorithms. FIG. 11 shows an example of real location data and synthetic location data extracted from the video training dataset under a common frame of reference when a user is making a first three times. This shows that the location data are similar and indicates the feasibility of synthesizing location data from a video training dataset. Similarly, FIG. 12 shows an example of real orientation data and synthetic orientation data extracted from the video training dataset under a common frame of reference. This also shows that the orientation data are similar and indicates the feasibility of synthesizing orientation data from a video training dataset.

It is contemplated that the domain of synthetic training data may be enhanced in size by performing data augmentation transformations. It is contemplated that performing data augmentation transformations may also increase the diversity of the dataset, thus making the machine learning models robust to extreme motion patterns, user diversity, variation in sensor position/orientation, etc.

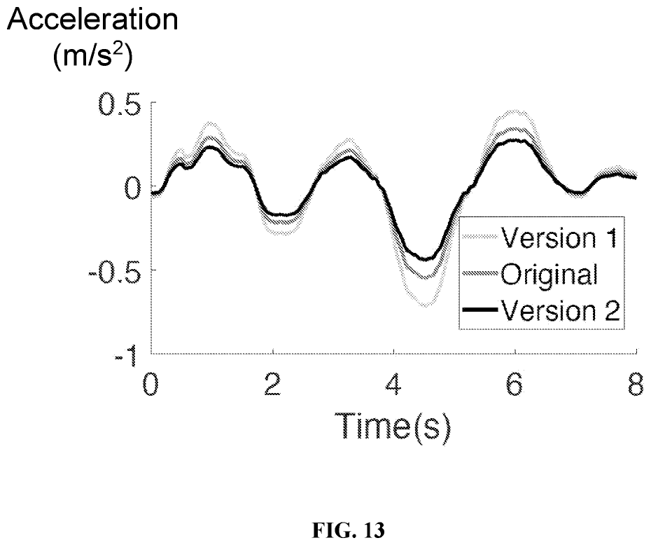

In some embodiments, a first data augmentation may be performed on the synthetic training data. The first data augmentation includes motion enhancement. It is contemplated that finger motion patterns extracted from the videos may be enhanced to vary the magnitude of flex/extensions and abduction/adductions. This may enhance the coverage of finger motion distribution to account for a large number or all possible configurations in practice. While performing this first data augmentation, finger motion constraints outlined below can be ensured (or defined), so that the augmented motion patterns are still realistic. FIG. 13 presents examples of such first data augmentation enhancements.

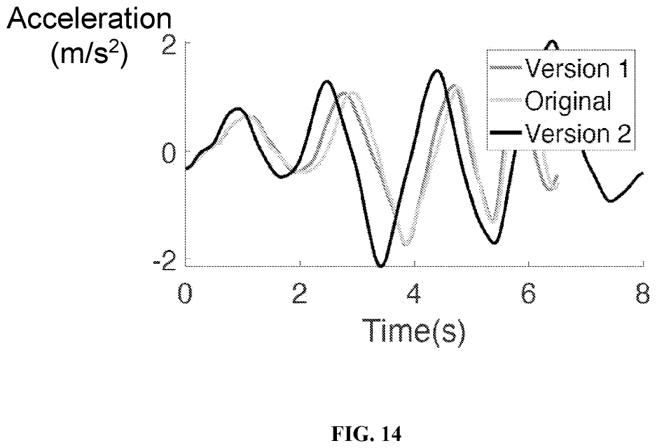

In some embodiments, a second data augmentation may be performed on the synthetic training data. The second data augmentation includes dynamic time warping (DTW). It is contemplated that (i) finger motion tends to be faster users when they are certain (otherwise, finger motion is slower, and (ii) users perform motion at different speeds and the speed of motion can vary with time. To emulate such diversity in real IMU data and make machine learning models robust at inference, DTW based augmentation may be used to create augmented versions of the original data. FIG. 14 depicts examples of such second data augmentation enhancements, wherein the original data being stretched and compressed.

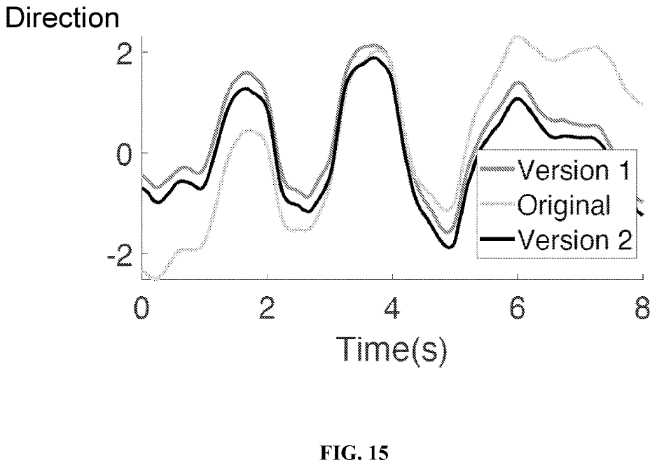

In some embodiments, a third data augmentation may be performed on the synthetic training data. The third data augmentation includes rotation augmentation. It is contemplated that sensor position/orientation might vary under regular usage, particularly when the user removes and remounts the sensor rings multiple times in a day. Thus, perturbations in the direction vectors in the training data to may be introduced to increase the diversity of sensor position/orientation. FIG. 15 shows an example wherein the third augmented data looks similar in shape to the original data but includes perturbations in the direction vectors.

As mentioned above, the known finger motion constraints can be ensured (or defined) while performing data augmentation so that the augmented motion patterns used to train the ML model are still realistic. For example, the four fingers (excluding the thumb) have four DoF from the following finger joints: (i) metacarpophalangeal (MCP) joint with two DoFs (Flex/extensions—φ_{mcp,f/e} and abduction/adductions φ_{mcp,aa}); (ii) proximal interphalangeal (PIP) joint (φₚᵢₚ) with a single DoF (Flex/extensions); and (iii) distal interphalangeal (DIP) (φ_{dip}) joint with a single DoF (Flex/extensions). The thumb has slightly different anatomy as its MCP and temporomandibular (TM) joints can both flex/extend and abduct/adduct, and its interphalangeal (IP) joint can only flex/extend. Thus, the thumb has five DoFs including φ_{mcp,f/e}, φ_{cp,a/a}, φ_{tm,f/e}, φ_{tm,a/a}, and φᵢₚ, forming a 21-dimension (R²¹) space of joint angles with the other four fingers. In addition to the fingers, wrist motion should also be accounted for. Wrist joint motion has three DoFs: (i) pronation/supination, φ_{wrist,p/s}; (ii) flexion/extension, φ_{wrist,f/e}; and (iii) radial/ulnar deviation, φ_{wrist,r/u}. Accordingly, the fingers and thumb have 21 DoFs whereas the wrist has 3 DoFs, totaling 24 DoFs.

It is contemplated that finger joints exhibit a high degree of correlation and interdependence. Equation (1) suggests that to bend the DIP joint, the PIP joint must also bend under normal finger motion (assuming no external force). Similarly, the range of motion for PIP is limited by the MCP joint.

$${ϕ}_{ip} = \frac{1}{2} {ϕ}_{mcp , f / e} , {ϕ}_{dip} = \frac{2}{3} {ϕ}_{pip} , {ϕ}_{mcp , f / e} = {k}_{ϕ pip} , 0 \leq k \leq \frac{1}{2} \qquad ( 1 )$$

Further, a range of motion constraints are outlined in Equation (2):

$$- 15 ° \leq {ϕ}_{mcp , aa} \leq 15 ° , 0 ° \leq {ϕ}_{dip} \leq 90 ° , 0 ° \leq {ϕ}_{pip} \leq 110 ° \qquad ( 2 )$$

In addition, there are complex interdependencies between joints of different fingers, which cannot be directly modeled by equations. However, the disclosed ML model is designed to automatically learn such constraints.

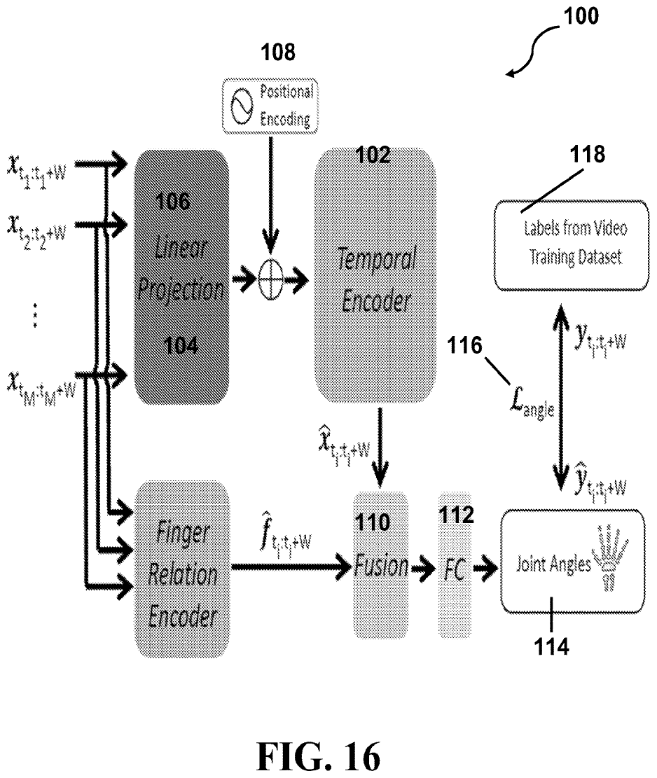

FIG. 16 shows an exemplary process for training a ML model 100 for three-dimensional finger motion tracking. The training process can include two branches: (i) a temporal encoder branch (Temporal Encoder 102) that exploits dependencies across time, and (ii) a finger relation encoder branch (Finger Relation Encoder 104) that exploits dependencies across fingers. It is contemplated that the temporal encoder branch 102 employs a transformer and self-attention mechanism that are efficient for processing series data (e.g., such as in natural language processing). It is further contemplated that the finger relation encoder branch 104 is designed on the observation that finger motions exhibit rich inter-finger correlation.

The input data used to train the ML model is represented by x∈ [symbol] ^{C×N}, where C=36 represents 6-dimensional input (acceleration and direction vectors) from sensors (fingers and wrist) and N denotes the number of time samples. To prepare input for the temporal encoder branch and the finger relation encoder branch, a sliding window to generate chunks M along the time axis is used, x_{ti:ti+W}=Chunk(x)∈ [symbol] ^{C×W}, i∈[1,M]. It is contemplated that W may be selected empirically. For example, W=700 may be selected.

Regarding the temporal encoder branch 102, to explore temporal dependencies, a transformer encoder may be used. The inputs, x_{ti:ti+W}, may be embedded by a linear projection layer 106 and then positional information may be added by a positional embedding layer 108, which are later utilized in the self-attention layer of the temporal encoder 102 to encode dependencies (across time) into the learned representations \hat{x}_{ti:ti+W}.

Regarding the finger relation encoder branch 104, it is contemplated that the design of this branch lies on a simple observation: finger motions are correlated as the motion of fingers can influence others (e.g., moving the middle finger most likely triggers the movement of the ring finger). To leverage the relation information across fingers, the following operations may be performed: given the input X_{ti:ti+W}∈ [symbol] ^{C×W}, we first reorganize the input X_{ti:ti+W}∈ [symbol] ^{C×W} into x′_{ti:ti+W}∈ [symbol] ^{C′×W′} where C′=6 and

$${W}^{′} = \frac{c}{{c}^{′}} \times W .$$

As wrist motion is independent of fingers, we may only keep the inputs from 5 fingers to get x″_{ti:ti+W}∈ [symbol] ^{(C′-1)×W′}. Secondly, we calculate the finger relation according to Equation (3).

$${f}_{ti : ti + W} = ( {x}_{tt : ti + w}^{″} ) ( {x}_{tt : ti + w}^{″} ) T ∈ {ℝ}^{( {c}^{′} - 1 ) \times ( {c}^{′} - 1 )} \qquad ( 3 )$$

Finally, the above information, f_{ti:ti+W}, may be incorporated into the model by a multi-layer perception module to obtain the representation for finger relations \hat{f}_{ti:ti+W}.

As seen in FIG. 16, the ML model may fuse representations from the temporal encoder branch 102 and the finger relation encoder branch 104 by an addition operation 110 and then a fully connected (FC) layer 112 to produce the final prediction of finger joint angles 114, represented by ŷ_{ti:ti+W}∈ [symbol] ^{18×W}. All chunks M (ŷ_{ti:ti+W} and y_{ti:ti+W}) may be linked to get \hat{φ}∈ [symbol] ^{18×N} and φ∈ [symbol] ^{18×N} for optimization. Among the 24 DoF finger joint angles discussed above, 18 joint angles are predicted by the model ( [symbol] ^{18×N}) whereas the rest are inferred.

The ML model 100 may further comprise a loss function ( [symbol] _{angle}) 116. It is contemplated that a loss function (i.e., error function) typically quantifies the difference between the difference between predicted outputs of a machine learning algorithm and the actual target values. The resulting value, the loss, reflects the accuracy of a model's predictions, and during training, a learning algorithm the gradient of the loss function with respect to the model's parameters to adjust these parameters and minimize the loss, effectively improving the model's performance on the dataset. In this case, the loss function 116 is configured to quantify the difference between the final prediction of finger joint angles 114 and the data extracted from the video training dataset 118.

It is contemplated that the mean square error (MSE) may be adopted as the main loss. The mean square error ( [symbol] ₘₛₑ) may be quantified according to Equation (4).

$${ℒ}_{mse} = {\sum }_{j = 1}^{j = 18} {( \hat{{ϕ}_{j}} - {ϕ}_{j} )}^{2} \qquad ( 4 )$$

In Equation (4), \hat{φ}ⱼ denotes the prediction 116 and φⱼ denotes the data extracted from the video training dataset 118. The \hat{φ}ⱼ's above include MSE errors for: (i) Four flex/extension angles of MCP joint from four fingers (excluding thumb) (ii) Four MCP adduction/abduction angles of the MCP joint from four fingers (\hat{φ}_{j,mcp,aa}∀ⱼ∈[1, 4]) (iii) Four PIP joint angles from four fingers (φ_{j,pip}∀ⱼ∈[1, 4]) (iv) Flex/extension and abduction/adduction angles of MCP and TM joints of the thumb, a total of four angles (φ_{mcp,f/e}, φ_{mcp,a/a}, φ_{tm,f/e}, φ_{tm,a/a}) (v) Flex/extension and Radial/ulnar deviation angles of the wrist (φ_{wrist,r/u}, φ_{wrist,f/e}). In addition, to encourage the similarity of the overall shapes of φⱼ and \hat{φ}ⱼ, a cosine similarity loss may be incorporated into the loss function as seen in Equation (5).

$${ℒ}_{cosine similarity} = {\sum }_{j = 1}^{j = 18} \frac{\hat{{ϕ}_{j}} \cdot {ϕ}_{j}}{max ( ‖ \hat{{ϕ}_{j}} ‖ 2 \cdot ‖ {ϕ}_{j} ‖ 2 , ϵ )} , ϵ = {10}^{- 6} \qquad ( 5 )$$

The overall loss function 114 is now expanded in Equation (6):

$${ℒ}_{angle} = {ℒ}_{mse} + α {ℒ}_{cosine similarity} \qquad ( 6 )$$

It is contemplated that a may be selected empirically. For example, α may be set to 0.4.

It is contemplated that the loss function 114 may not include φ_{dip} or φᵢₚ, as they may be computed directly from known finger motion constraints described above. The pronation/supination angle (φ_{wrist,p/s}) for the wrist may also be directly computed from the orientation of the sensor.

It is contemplated that the disclosed method, and particularly the motion evaluation platform and trained ML model, may exploit correlations across fingers and time to track 3D finger motion with a joint accuracy of 6.57 mm and a joint angle accuracy of 8.68 degrees. It is further contemplated that this accuracy is consistent over all finger joints and various users.

It is contemplated that such three-dimensional finger tracking may enable exciting applications in the areas of augmented and virtual realities (AR and VR), sports analytics, sign language recognition and translation, smart healthcare, among other applications. It is further contemplated that in contrast to camera-based approaches that can be privacy sensitive and reliant on adequate lighting and resolution, disclosed embodiments are privacy agnostic and function in all environments, including when the user is constantly moving.

### Blood Alcohol Concentration (BAC) Measurement

As explained above, sensor data may be transmitted from at least one ring 10 to the input/output device 40 and/or server 42 for analysis, evaluation, etc. In exemplary embodiments, the sensor array 30 comprises a PPG sensor 30a, an IMU sensor 30b, and a thermal sensor 30c. In such embodiments, the PPG sensor 30a may track changes in light absorption caused by changes of blood vessels, the IMU sensor 30b may track a user's activity, such as the position, orientation, and motion of a user's ring, and the thermal sensor 30c may track the skin contact temperature of the ring, and the microcontroller 24 may transmit this data to the input/output device 40 and/or server 42. It is contemplated that this data may provide continuous BAC monitoring and gather insight into alcohol impact on human body in a non-invasive and passive approach. Compared with other sensing methods such as bodily fluid testing or breath alcohol concentration (BrAC), this embodiment is much more convenient, non-invasive, calibration-free and provides better user experience without user effort.

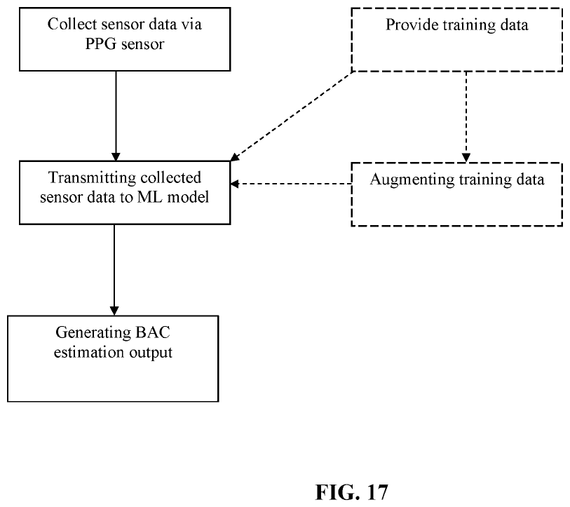

As seen in FIG. 17, an exemplary method of monitoring BAC therefore includes capturing sensor data using at least the PPG sensor 30a of a ring 10. For example, it is contemplated that alcohol consumption may lead to dilation on blood vessels due to alcohol's effects on the body's cardiovascular system. It is therefore contemplated that the sensor data may include changes in light absorption caused by changes of blood vessels captured with the PPG sensor 30a. The input/output device 40 and/or the server 42 may then estimate volumetric changes in the blood by analyzing the changing pattern of blood vessels/flow using the collected data.

The method further includes transmitting the data to a BAC evaluation platform. The BAC evaluation platform may be configured to process the collected sensor data to generate a BAC estimation output based on the sensor data from the ring(s) communicated to that platform. For example, the BAC evaluation platform may include a pre-defined BAC determination processing mechanism to process the collected data. The BAC evaluation platform may be hosted on an input/output device 40 and/or a server 42.

In some embodiments, the pre-defined BAC determination processing mechanism may implement a trained machine learning (ML) model. The ML model may be hosted on an input/output device 40 (e.g. a smart phone, a laptop computer, a tablet, etc.) and/or a server 42 that may be communicatively connected to the input/output device 40 (e.g. via an application programming interface, API, or other communicative connection that may be provided via at least one network such as the internet, etc.). It is contemplated that the ML model uses data together with machine learning algorithms that undergo a training process to prepare a trained ML model. In this case, the ML model may use a predetermined training dataset and machine learning algorithms to prepare a trained ML model that may utilized by the BAC evaluation platform in processing collected data to generate a BAC estimation output.

It is contemplated that a domain of training data may be enhanced in size by performing data augmentation transformations. It is contemplated that performing data augmentation transformations may also increase the diversity of the dataset, thus making the machine learning models robust to user diversity, variation in sensor position/orientation, etc.

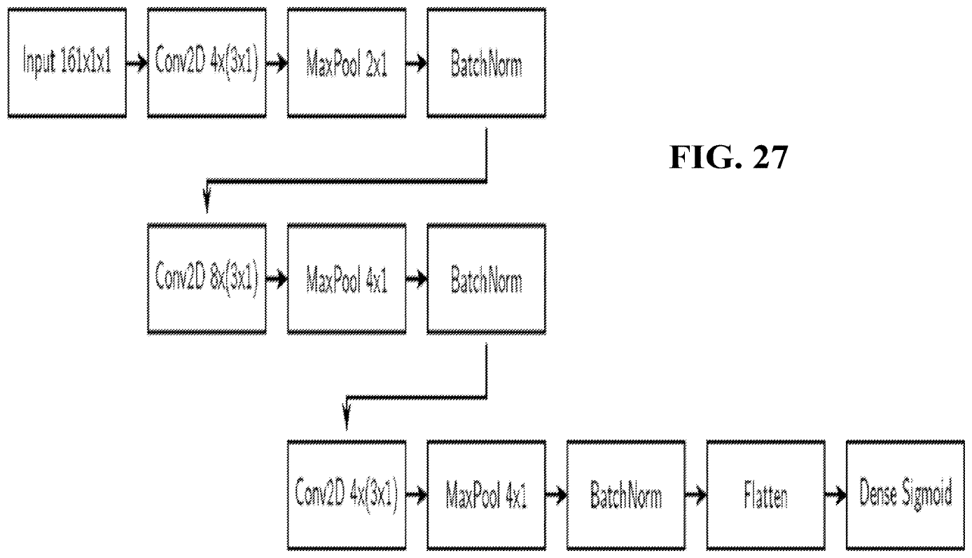

It is contemplated that the ML model may comprise a convolutional neural network(s) to overcome limited training data. As seen in FIG. 27, the network may consist of three main blocks, each comprising a convolutional layer with padding to maintain dimensionality, followed by a max pooling layer to reduce dimensions, and a batch normalization layer to stabilize learning. The first block uses 4 filters, and the second and third use 8 and 4 filters respectively. After these blocks, the network flattens the outputs to connect to a dense layer equipped with a single neuron and a sigmoid activation function.

The disclosed method, and particularly the BAC evaluation platform and trained ML model, may exploit the changes of PPG waveform due to alcohol to predict BAC with a mean absolute error of 0.0069.

### EXAMPLES

### Example 1: Exemplary Ring Construction and Components

An exemplary ring was constructed according to the disclosure provided herein. Various hardware components were carefully assembled into a double-sided flexible printed circuit board as described above to fit low form factor requirements. We designed a NRF52832 microcontroller unit which interfaces with the rest of the electronics based on the design of a Sparkfun nRF52832 Breakout. The microcontroller unit consisted of a 2.4 30 GHz radio frequency (RF) transceiver for BLE and ARM Cortex-M4 32-bit 64 MHz processor with floating-point unit (FPU) and supported multiple interfaces such as SPI, I²C, and UART. Such an SoC-based design choice resulted in a miniature microcontroller unit (e.g., 6 mm×6 mm). The IMU sensor chip incorporated was ICM20948 which provided 9-axis IMU data and interfaced with the microcontroller unit using SPI. The PPG sensor incorporated was Maxim Integrated MAX30101 which consisted of three light emitting diodes (IR LEDs, Red LEDs, and Green LEDs) and a photodiode, and provided data using I²C. The whole printed circuit board was powered with a 15 mAh, 3.7V ring-shaped LiPo battery with a radius of 10.55 mm and thickness of 1.6 mm, which easily fit into the shape of a ring. Because the microcontroller unit required 3.3 V whereas the IMU and PPG sensors require 1.8 V, we used an advanced dual low-drop-out (LDO) voltage regulator (MIC5370) for providing two independently controlled power sources. Finally, we also integrated a circuit to protect PCB and battery from short current or overcharging. Overall, the power consumption of the hardware was about 12 mA when actively streaming the sensor data, and only about 76 μA under low power mode while the microcontroller unit was still collecting data and only streams the data periodically to the smartphone app. This provided sufficiently low power design to support a battery life of a week, thus enabling applications like sleep monitoring. In order to conveniently charge the ring, we designed a charger. The charger is connected to USB power source via the USB micro B cable, with charge rate of 1 C.

A second exemplary ring was constructed according to the disclosure provided herein. The second exemplary ring was substantially similar to the exemplary ring described above, but further included an OLED display interfacing with the microcontroller via 3-wire SPI. The OLED display was 160×32 flexible gray-scale OLED Display y Crystalfontz, which provided 160×32 4-bit grayscale pixels with 88 dots-per-inch (DPI) and a 3-wire SPI interface. Because MCU, IMU, PPG and temperature sensors required 3.3V and 1.8V where display required 12V supply, a dual low-drop-out (LDO) voltage regulator MIC5370 by Microchip Technology was employed to supply MCU and sensing units and step-up DC-DC converter AP3012KTR y Diodes Incorporated to boost up battery voltage to 12V to power OLED display. Finally, a battery management unit was implemented to protect the PCB and battery from overcharging. A curved lithium battery with 60 mAh was employed. Overall, the power consumption of the hardware was about 29.5 mA when actively visualizing sensing data on OLED display and streaming data to mobile device, enabling up to two hours of continuous display usage.

### Example 2: User Experience Study

We conducted a user experience study based on the ring as designed above in Example 1.

The study included participants who wore the ring with embedded sensors continuously under free-living conditions. Each of twelve users wore several alternative, comparative platforms to the ring and compared the platforms with each other. We compared the ring with three other finger-based sensing or typing devices (Comparative Platform 1—Tapstrap, Comparative Platform 2—Myo, and Comparative Platform 3—CyberGlove). The users conducted normal daily life activities such as working on their laptops (typing, browsing, etc.), eating, drinking, watching movies, etc., while wearing the ring. Participants rated the four devices anonymously based on comfort, weight, and appearance from 0 to 10. Comparative Example 1 turned out to be rigid and not comfortable for long-time wearing and Comparative Example 2 precluded finger motion that requires precision (cooking, typing, etc.) apart from causing sweat. While Comparative Example 3 received higher ratings than these two, users reported it as heavy and less comfortable for long-time wearing with restricted mobility of fingers due to the straps. The ring as described above secured the highest scores in comfort, weight, and appearance as shown in Table 1.

TABLE 1
Results of user experience survey

| | Comfort | Weight | Appearance |
|---|---|---|---|
| Platform | Score | Score | Score |
| Embodiment of Present Application | 9.5 | 9.8 | 9.7 |
| Comparative Example 1 | 5.3 | 6.6 | 7.4 |
| Comparative Example 2 | 8.0 | 6.4 | 3.6 |
| Comparative Example 3 | 4.2 | 2.2 | 6.4 |

### Example 3: Three-Dimensional Finger Motion Tracking

We conducted a three-dimensional finger motion tracking study based on the ring as designed above in Example 1.

Data Collection Methodology: Since there is no publicly available IMU dataset for finger motion tracking, we collected our own dataset using the technique described above in the description (e.g., training data was extracted from sources of online videos). We conducted a study with 12 users (8 males, 4 females), aged between 20-47 and weighing between 44-105 kgs. For stress testing of the ring across all possible hand poses, we covered an exhaustive space of all possible hand poses. The majority of all possible hand poses are known to be one of a base state or transitioning between these base states based on anatomical feasibility constraints.

The users were allowed to make natural and random finger motions while ensuring that base states were included in their motion pattern in random order. This can help ensure good coverage by incorporating the entire range of motion of possible hand poses. Each user participates in 5 sessions of 5 mins while removing and remounting the sensor across sessions to validate robustness to natural changes in sensor positions and orientation. Wrist and arm mobility was varied across sessions. The accuracy was also studied for longer duration free-living experiments.

Labels for Validation: The collected data included 9-axis IMU and the fingers' 3D coordinates and joint angles were captured by a Leap depth camera. The IMU data provides motion data for 3D finger tracking. The Leap data served as ground truth. We note that Leap has location tracking error <0.2 mm, thus sufficient for ground truth. Since the ring performs continuous finger tracking instead of discrete gesture classification, we employ the loss function defined in Equation (6).

Implementation: The ML model was implemented with Pytorch. The training was done on a desktop with Intel i7-8700K CPU, 32 GB RAM, and an NVIDIA RTX 2080 GPU. We used Adam optimizer (β1 of 0.9, β2 of 0.98), and a varied learning rate based on literature. To avoid overfitting, we applied the L2 regularization (parameter of 0.01) and a dropout rate (parameter of 0.5) following each layer. Once trained, the inference was done on smartphones (Samsung S20, OnePlus 9 Pro) using Pytorch Mobile.

Motion Tracking Performance: To assess the performance of the disclosed ring, we conducted the following analysis: (i) since no public datasets exist for IMU-based 3D finger motion tracking, we performed a comparison with naive baselines including ablation studies. This validated the importance and effectiveness of design choices of the disclosed ring; (ii) we conducted robustness studies to characterize the performance of the disclosed ring over accuracy vs fingers, users, number of sensors, and long durations (ability to handle sensor drifts); (iii) we provided a qualitative reconstruction of hand poses and compare the results to the ground truth; (iv) we evaluated power consumption and latency on smartphones when executing ML models; (v) we contrasted features and accuracy of the disclosed ring with prior work.

Ablation Study of Baselines: We converted joint angle errors to position errors and present position error (millimeters) for most results. FIG. ##shows the disclosed ring against three baselines. (1) CNN-LSTM-based Model: We built a CNN-LSTM-based model for the same tracking problem to verify that the design of the disclosed ML model was effective in capturing temporal relations for time-series data. In comparison to this design, we observed that the disclosed ring reduces the error by 20.4% and 23.2% for median and mean location error respectively. (2) w/o Cosine Similarity Loss: The results showed, by encouraging similar shapes of the output and the ground truth with Equation (5), the location error decreases by 16.4% and 9.2% for median and mean respectively. (3) w/o Finger Relation Encoder: The finger relation encoder branch as described above aims to take finger relations into consideration. To test its effectiveness in tracking 3D finger motions, we built a model without this module. FRE improves the accuracy by 20.4% and 18.2% for median and mean location error respectively.

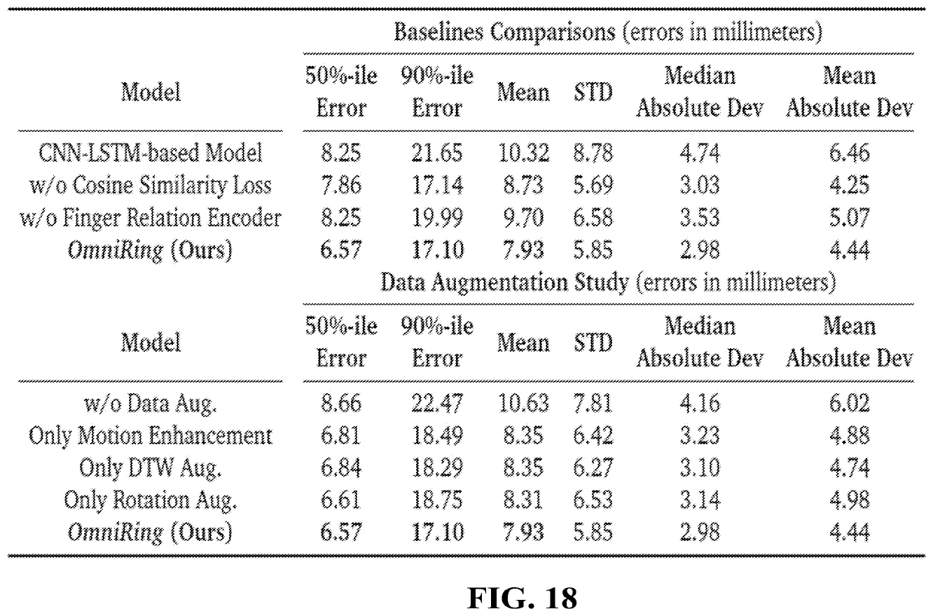

Ablation Study of Data Augmentation: We also studied the effectiveness of individual data augmentations in the disclosed ring as shown in FIG. 18 (1) Motion Enhancement decreased mean location error by 5%, which shows the effectiveness of increasing the coverage of finger motion distribution in reality. (2) DTW Augmentation also improved performance by a similar degree, by incorporating different speeds of different parts of finger motion into the training data. (3) Rotation Augmentation emulated the diversity of wearing positions in daily life, and improved the disclosed ring by 4.6% for mean location error. Overall, the combination of all three data augmentation techniques boosted the performance of the ring by 24.1% and 23.2% for median and mean location errors respectively, which demonstrates that the ring can acquire knowledge from a different domain with the help of simple but effective data augmentation methods, and henceforth mitigate the need of large-scale IMU datasets.

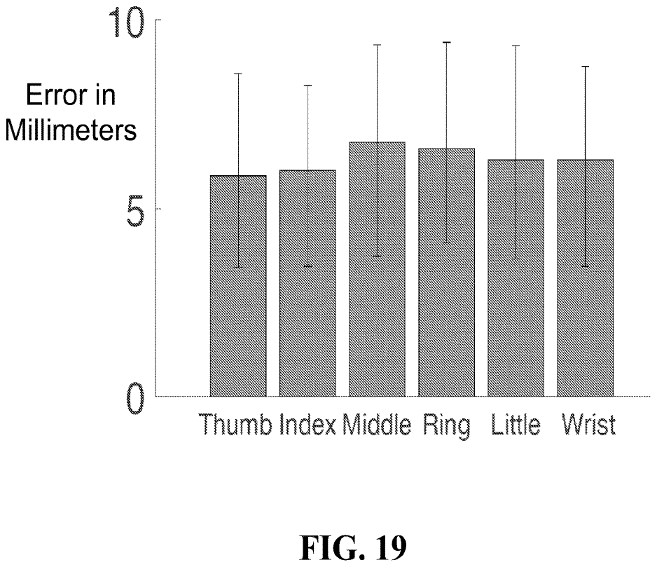

Robustness Study—Accuracy vs. Fingers and Wrist: As seen in FIG. 19, synthetic training data and data augmentation transformations incorporated the full range of motion across fingers and wrist, enabling accurate tracking of all fingers and wrist.

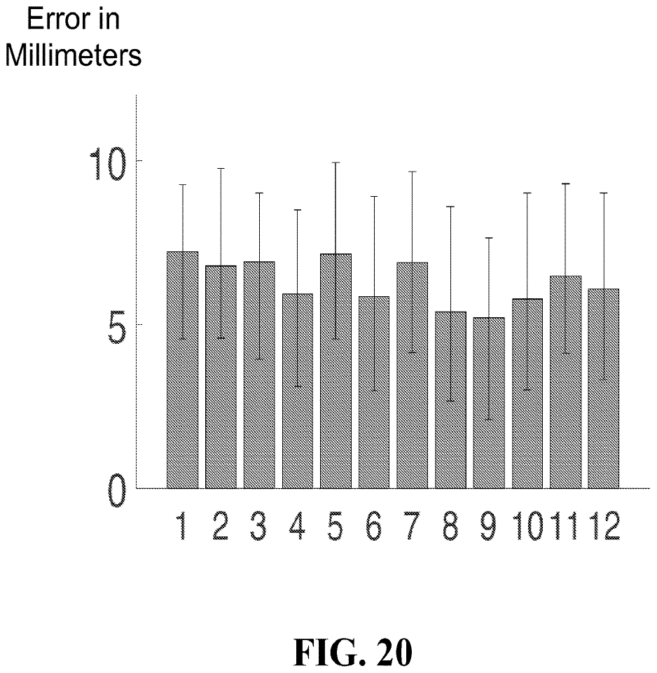

Robustness Study—Accuracy vs. Users: As seen in FIG. 20, the ring achieved consistent accuracy across users with minor variations. The variation happened because some users perform faster motions. Nevertheless, the user with the worst performance only has ˜1 mm (1.35°) higher error than average. Given the diverse sources from which the synthetic training data was generated with the added diversity via data augmentations, we believe the ring generalizes to a variety of users.

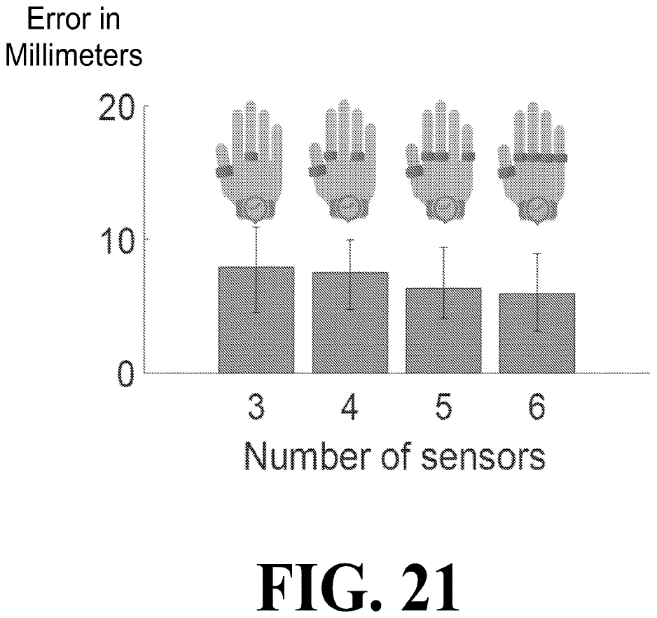

Robustness Study—Accuracy vs. Number of Sensors: As seen in FIG. 21, the position of sensors for each case is also indicated. There is a graceful degradation in accuracy with fewer sensors. With a high degree of interaction between fingers, the motion of one finger will cause other fingers to move, thus enabling tracking them even without sensor rings on all fingers, which can further enhance the comfort levels of wearing.

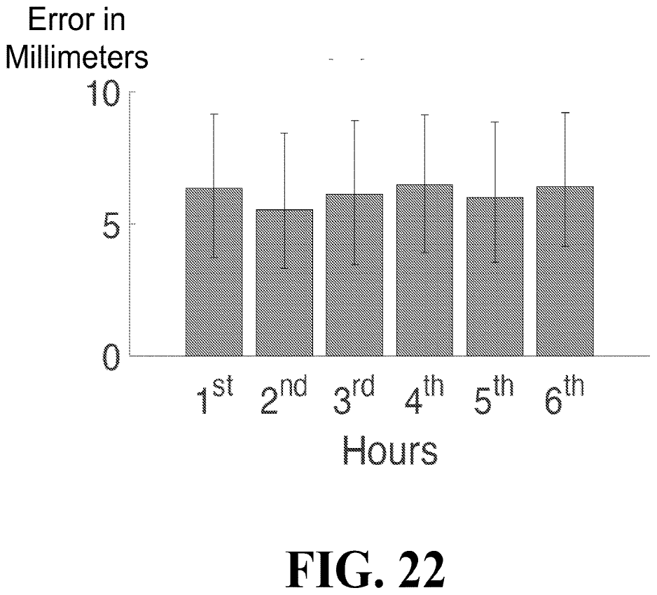

Longer Session Experiments (Free-Living Conditions): We conducted free-living experiments to study long-term effects like potential drifts. Users were instructed to wear the sensor continuously for 6 hours at their apartments. At the end of each hour, we conducted a 5-minute session of finger motion as per our user study protocol. In between sessions, the users conducted daily activities including working on their laptops (typing, browsing, etc.), eating, drinking, watching movies, etc., while wearing the sensor. The results are shown in FIG. 22. As expected, the accuracy does not degrade with time. This is because the ring does not perform long-term integration of data, which is the main source of drift errors. In contrast, the ring opportunistically resets drifts, and eliminates magnetic interference.

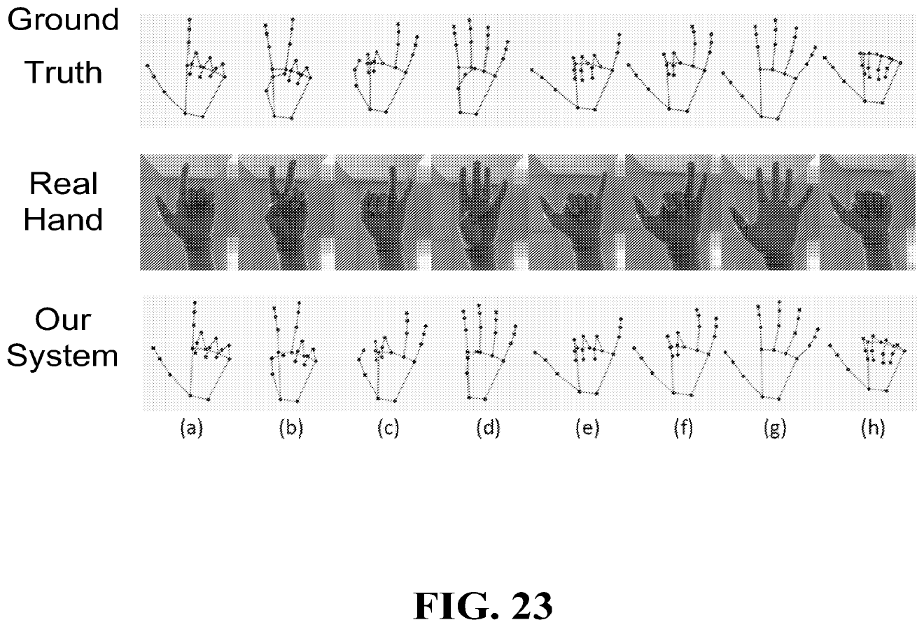

Qualitative Results: FIG. 23 shows qualitative results. The figure compares the tracking of hand poses by the ring with reference to the real hand and ground truth (from Leap). The ring can capture a wide range of finger motions with decent accuracy, even without the need to place sensor rings on all fingers by exploring the inter-finger correlations. Overall, we believe these results are promising in the context of applications in activity tracking, sport analytics, etc.

Power Consumption and Latency: To make the ring real-time, at any given instant of time, we feed previous few chunks to the ML model. The latency of execution on Samsung S20 and OnePlus 9 Pro are around 8.4 ms and 7.6 ms respectively, sufficient for real-time applications. The real-time power discharge rate is 14.43% and 16.72% per hour for Samsung S20 and OnePlus 9 Pro, while the discharge rate under the low-power mode is around 6% for both models.

### Example 4: Heart Rate Estimation

We conducted basic feasibility of extraction of raw PPG data from the ring for as designed above in Example 1.

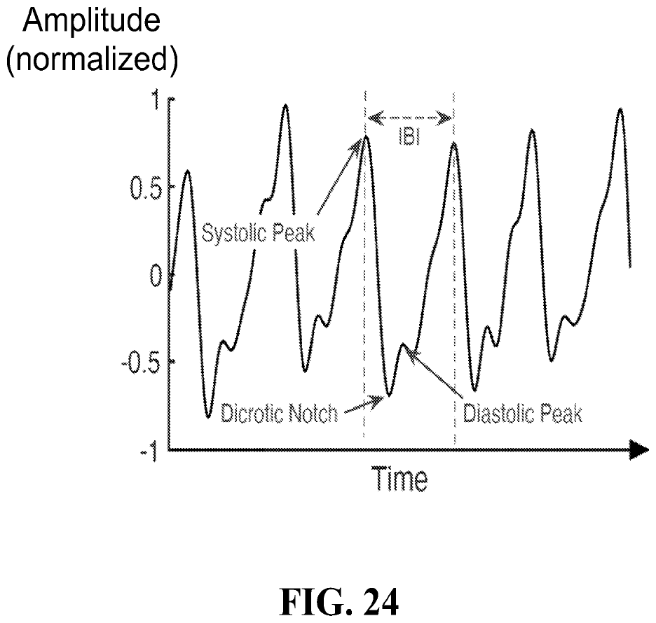

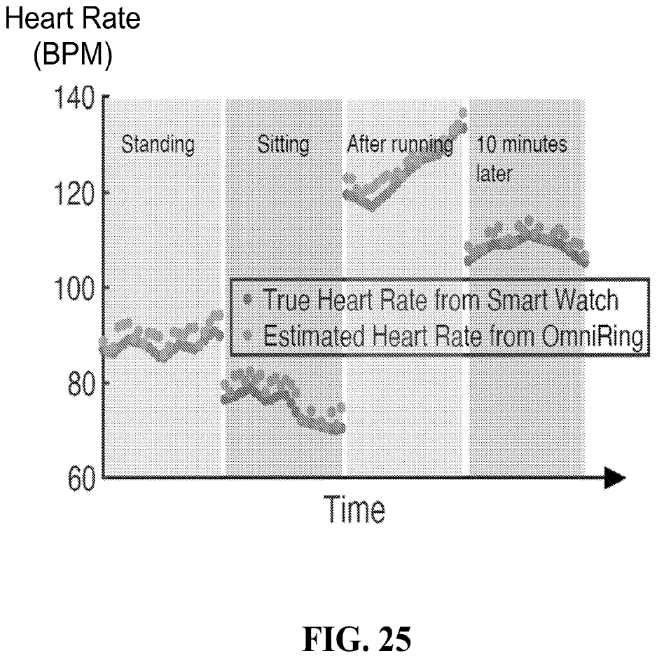

In particular, the ring is incorporated with a PPG sensor for health related sensing tasks such as heart rate, SpO₂, etc. For example, a PPG sensor illuminates the skin and measures the absorption which can be used for detecting blood volume changes in the subcutaneous tissues of the skin due to the pumping of blood during the cardiac cycle. Therefore, by measuring the absorption, metrics like heart rate and blood pressure can be computed. To validate the PPG data, we conducted a preliminary study for heart rate monitoring, in which we asked users to perform simple activities (i.e., standing, sitting, running, and standing) and collected PPG data for estimating the heart rates. FIG. 24 shows an example of PPG data that is processed by a bandpass filter and biometric characteristics (e.g., Systolic/Diastolic peaks, IBI, etc.) denoted in the figure can be utilized to monitor heart rates. We estimated the heart rates when users remain static after each activity and FIG. 25 depicts the ring monitors heart rates closely.

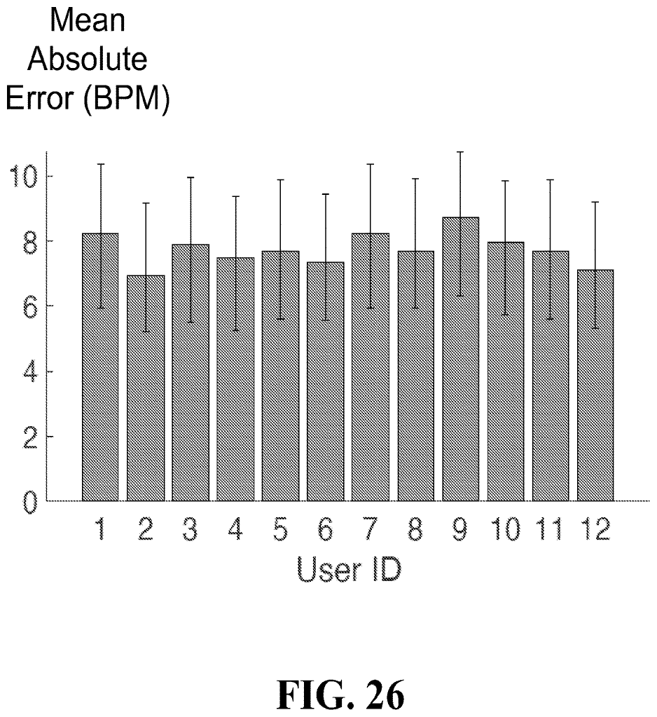

In addition, we studied heart rate monitoring accuracy for individuals as shown in FIG. 26. Overall, the heart rate estimation is stable across different users. It is worth noting that our users not only span a various range of ages and body shapes but also cover different skin tone colors. However, we don't see significant difference between light and dark skin tones. We believe this preliminary study has demonstrated that the ring has the potential to enable healthcare applications in addition to motion tracking.

### Example 5: Blood Alcohol Concentration Measurements

We conducted a blood alcohol concentration measurement study based on the ring as designed above in Example 1. However, the ring further included a thermal sensor. The thermal sensor incorporated is TMP117 which provided high-precision digital temperature of the finger using I²C. The combination of PPG, IMU and thermal sensors provided insight into alcohol impact on human body in a non-invasive and passive approach. Compared with prior sensing methods such as bodily fluid testing or breath alcohol concentration (BrAC), the ring is much more convenient, non-invasive, calibration-free and provides better user experience without user effort.

Evaluation Methodology: To measure the accuracy of the proposed BAC estimation method, we utilized the metric mean absolute error (MAE), which is widely accepted by other bio-signal estimation work, such as BP, breath rate, tidal volume.

Mean Absolute Error: We evaluated the estimation MAE based on user-dependent training, which means that both training and testing data (80/20 split) were from the same user.=Overall, the ring achieved decent performance across all 15 participants, with a mean MAE at 0.00697 with maximal MAE 0.0081. This result indicates that our system can effectively estimate the performance of our system.

It should be understood that modifications to the embodiments disclosed herein can be made to meet a particular set of design criteria. For instance, the number of or configuration of components or parameters may be used to meet a particular objective.

It will be apparent to those skilled in the art that numerous modifications and variations of the described examples and embodiments are possible in light of the above teachings of the disclosure. The disclosed examples and embodiments are presented for purposes of illustration only. Other alternative embodiments may include some or all of the features of the various embodiments disclosed herein. For instance, it is contemplated that a particular feature described, either individually or as part of an embodiment, can be combined with other individually described features, or parts of other embodiments. The elements and acts of the various embodiments described herein can therefore be combined to provide further embodiments.

It is the intent to cover all such modifications and alternative embodiments as may come within the true scope of this invention, which is to be given the full breadth thereof. Additionally, the disclosure of a range of values is a disclosure of every numerical value within that range, including the end points. Thus, while certain exemplary embodiments of the apparatus and process and/or utilization and methods of making and using the same have been discussed and illustrated herein, it is to be distinctly understood that the invention is not limited thereto but may be otherwise variously embodied and practiced within the scope of the following claims.

1. (canceled)

2. A method of tracking three-dimensional finger motion, comprising:
providing one or more wearable rings comprising a microcontroller unit, a non-transitory computer readable medium connected to the microcontroller unit, a Bluetooth transceiver unit connected to the microcontroller, and an inertial measurement unit (IMU) sensor;
detecting motion of at least one of the one or more wearable rings via a motion detection module;
collecting motion data via the IMU sensor;
providing the motion data to a motion evaluation platform; and
processing the motion data using a motion determination processing mechanism implementing a trained machine learning model to generate a three-dimensional hand pose output, wherein the trained machine learning model comprises a temporal encoder branch and a finger relation branch.

3. The method of claim 2, wherein the temporal encoder branch is trained by:
feeding synthesized training data to the temporal encoder branch to generate a temporal output represented by the formula \hat{x}_{ti:ti+W},
wherein \hat{x} is the temporal output, tᵢ is any integer greater than or equal to one, and W is any empirically selected value.

4. The method of claim 3, wherein the synthesized training data is augmented prior to being fed to the temporal encoder branch.

5. The method of claim 2, wherein the finger relation encoder branch is trained by:
feeding synthesized training data to the finger relation encoder branch to generate a finger relation output represented by the formula \hat{f}_{ti:ti+W},
wherein \hat{f} is the finger relation output, tᵢ is any integer greater than or equal to one, and W is any empirically selected value.

6. The method of claim 5, wherein the synthesized training data is augmented prior to being fed to the finger relation encoder branch.

7. The method of claim 2, wherein the temporal encoder branch and the finger relation encoder branch are fused to produce a final prediction of finger joint angles represented by the formula ŷ_{ti:ti+W}∈ [symbol] ^{18×W},
wherein ŷ is the final prediction of finger joint angles, tᵢ is any integer greater than or equal to one, and W is any empirically selected value.

8. The method of claim 2, wherein the trained machine learning model further comprises a loss function comprising a mean square error loss and a cosine similarity loss.

9. The method of claim 8, wherein the mean square error loss is represented by the formula
$${ℒ}_{mse} = {\sum }_{j = 1}^{j = 18} {( \hat{{ϕ}_{j}} - {ϕ}_{j} )}^{2} ,$$
wherein \hat{φ}ᵢ represents predicted finger joint angles and φᵢ represents joint angles extracted from synthesized training data.

10. The method of claim 8, wherein the cosine similarity loss is represented by the formula
$${ℒ}_{cosine similarity} = {\sum }_{j = 1}^{j = 18} \frac{\hat{{ϕ}_{j}} \cdot {ϕ}_{j}}{max ( ‖ \hat{{ϕ}_{j}} ‖ 2 \cdot ‖ {ϕ}_{j} ‖ 2 , ϵ )} ,$$
wherein \hat{φ}ᵢ represents predicted finger joint angles, φᵢ represents joint angles extracted from synthesized training data, and ε is 10⁻⁶.

11. The method of claim 2, further comprising:
providing a first wearable ring, a second wearable ring, and a third wearable ring, each wearable ring comprising a microcontroller unit, a non-transitory computer readable medium connected to the microcontroller unit, a Bluetooth transceiver unit connected to the microcontroller, and an IMU sensor.

12. The method of claim 11, wherein the first wearable ring is positioned on a thumb of a user, the second wearable ring is positioned on an index finger of the user, and the third wearable ring is positioned on a ring finger of the user.

13-15. (canceled)

16. The method of claim 2, wherein the one or more wearable rings further comprises an organic light-emitting diode display.

17. The method of claim 16, further comprising:
displaying the motion data via the display to a user;
displaying the three-dimensional hand pose output via the display to a user; or
displaying messages configured to communicate information via the display to a user.

18. (canceled)

19. A system for tracking three-dimensional finger motion, comprising:
one or more wearable rings comprising a microcontroller unit, a non-transitory computer readable medium connected to the microcontroller unit, a Bluetooth transceiver unit connected to the microcontroller, and an IMU sensor, wherein the IMU sensor is configured to collect motion data;
a motion detection module configured to detect motion of at least one of the one or more wearable rings; and
a motion evaluation platform comprising a motion determination processing mechanism configured to process the motion data to generate a three-dimensional hand pose output, wherein the motion determination processing mechanism implements a trained machine learning model, wherein the trained machine learning model comprises a temporal encoder branch and a finger relation branch.

20. The system of claim 19, wherein the temporal encoder branch is trainable via synthesized training data and configured to generate a temporal output represented by the formula \hat{x}_{ti:ti+W},
wherein \hat{x} is the temporal output, tᵢ is any integer greater than or equal to one, and W is any empirically selected value.

21. The system of claim 19, wherein the finger relation encoder branch is trainable via synthesized training data and configured to generate a finger relation output represented by the formula \hat{f}_{ti:ti+W},
wherein \hat{f} is the finger relation output, tᵢ is any integer greater than or equal to one, and W is any empirically selected value.

22. The system of claim 19, wherein the temporal encoder branch and the finger relation encoder branch are configured to be fused to produce a final prediction of finger joint angles represented by the formula ŷ_{ti:ti+W}∈ [symbol] ^{18×W},
wherein ŷ is the final prediction of finger joint angles, tᵢ is any integer greater than or equal to one, and W is any empirically selected value.

23. The system of claim 19, wherein the trained machine learning model further comprises a loss function comprising a mean square error loss and a cosine similarity loss.

24. The system of claim 23, wherein the mean square error loss is represented by the formula
$${ℒ}_{mse} = {\sum }_{j = 1}^{j = 18} {( \hat{{ϕ}_{j}} - {ϕ}_{j} )}^{2} ,$$
wherein \tilde{φ}ᵢ represents predicted finger joint angles and φᵢ represents joint angles extracted from synthesized training data.

25. The system of claim 23, wherein the cosine similarity loss is represented by the formula
$${ℒ}_{cosine similarity} = {\sum }_{j = 1}^{j = 18} \frac{\hat{{ϕ}_{j}} \cdot {ϕ}_{j}}{max ( ‖ \hat{{ϕ}_{j}} ‖ 2 \cdot ‖ {ϕ}_{j} ‖ 2 , ϵ )} ,$$
wherein \tilde{φ}ᵢ represents predicted finger joint angles, φᵢ represents joint angles extracted from synthesized training data, and ε is 10⁻⁶.

26. The system of claim 19, comprising:
a first wearable ring, a second wearable ring, and a third wearable ring, wherein the first wearable ring is positioned on a thumb of a user, the second wearable ring is positioned on an index finger of the user, and the third wearable ring is positioned on a ring finger of the user.

27-29. (canceled)

30. The system of claim 19, wherein the one or more wearable rings further comprises an organic light-emitting diode display.

31. The system of claim 30, wherein the display is configured to
display the motion data to a user;
display the three-dimensional hand pose output to a user; or
display messages configured to communicate information to a user.

32-44. (canceled)
